(19) United States
(12) Patent Application Publication
(10) Pub. No.: US 2026/0356800 A1
(43) Pub. Date: Nov. 12, 2026

(54) POLYMORPH OF PHENYL PYRROLE HYDRAZINE CARBOXIMIDAMIDE COMPOUND

(71) Applicant: SYNACT PHARMA ApS, Holte (DK)

(72) Inventors: Thomas BOESEN, Copenhagen Ø (DK); Thomas Engelbrecht Nordkild JONASSEN, Holte (DK); Hayley Ann REECE, Edinburgh (GB); Ross MCLELLAN, Glasgow (GB)

(21) Appl. No.: 19/140,098

(22) PCT Filed: Dec. 21, 2023
(86) PCT No.: PCT/EP2023/087213

§ 371 Date: Jun. 17, 2025

(30) Foreign Application Priority Data
Dec. 21, 2022 (EP) 22215369.4

(51) Int. Cl. C07D 207/335; A61K 31/402

(52) U.S. Cl. CPC C07D 207/335; A61K 31/402

(57) ABSTRACT

The present disclosure relates to a crystalline form of 2-{3-[1-(2-nitrophenyl)-1H-pyrrol-2-yl]prop-2-en-1-ylidine}hydrazine-1-carboximidamide having high solubility at acidic conditions. The disclosure also relates to use of said crystalline form in medicine.

### TECHNICAL FIELD

The present invention relates to a polymorph of 2-{3-[1-(2-nitrophenyl)-1H-pyrrol-2-yl]prop-2-en-1-ylidine}hydrazine-1-carboximidamide having high solubility at low pH, and formulations comprising same.

### BACKGROUND

The melanocortin system is a set of neuropeptidergic and immuneendocrine signalling pathways that play an integral role in the homeostatic control of a diverse array of physiological functions, including melanogenesis, stress response, inflammation, immunomodulation and adrenocortical steroidogenesis. It consists of multiple components, including the five G protein-couple melanocortin receptors: melanocortin receptor 1 (MC1R) to MC5R; peptide ligands; α, β, γ-melanocyte stimulating hormone (α, β, γ-MSH); adrenocorticotropic hormone (ACTH) secreted by the anterior pituitary; and endogenous antagonists. The biological functions of the melanocortin system are mediated by the five melanocortin receptors (MCRs), which have distinct tissue distribution, convey different signalling and exert varying biological activities in different organ systems.

Phenyl pyrrole hydrazine carboximidamide derivatives with activity on the melanocortin receptors have previously been disclosed. One example of such compound is the anti-inflammatory AP1189 (2-{3-[1-(2-nitrophenyl)-1H-pyrrol-2-yl]prop-2-en-1-ylidine}hydrazine-1-carboximidamide) which was first shown to bind the MC1R and later was identified as a biased dual agonist at receptors MC1R and MC3R that does not provoke canonical cAMP generation (and hence no MC1R-induced melanogenesis) but instead appear to induce alternative pathways including ERK1/2-phosphorylation and Ca^{2±} mobilisation.

### SUMMARY

The present inventors have discovered a polymorph of AP1189 with a particularly favourable solubility profile. The inventors found this polymorph of AP1189 is highly soluble at acidic conditions, contrary to some polymorph salt forms of AP1189.

One aspect of the present disclosure provides for a crystalline form of 2-{3-[1-(2-nitrophenyl)-1H-pyrrol-2-yl]prop-2-en-1-ylidine}hydrazine-1-carboximidamide (AP1189, free base) exhibiting at least X-ray lines (2-theta values) in a powder diffraction pattern when measured using Cu K_{α} radiation at 14.5±0.2, 23.9±0.2, and 26.7±0.2 (AP1189 Pattern 1).

The present disclosure also provides methods of producing such crystalline form.

One aspect of the present disclosure provides for a method for producing a crystalline form of AP1189 of Pattern 1, said method comprising:
i. mixing AP1189 and a solvent to form a mixture; and
ii. isolating the crystalline form of AP1189 of Pattern 1 from said mixture.

One aspect of the present disclosure provides for a method for producing the crystalline form of AP1189 of Pattern 1, said method comprising:
i. mixing an AP1189 salt and a solvent to form a composition; and
ii. isolating the crystalline form of AP1189 of Pattern 1 from said composition.

One aspect of the present disclosure provides for a method for producing the crystalline form of AP1189 of Pattern 1, said method comprising:
i. mixing 3-[1-(2-nitrophenyl)-1-H-pyrrole-2-yl]-propanal and amino guanidine or a salt thereof in a solvent to form a composition, and
ii. isolating the crystalline form of AP1189 of Pattern 1 from said composition.

One aspect of the present disclosure provides for a method for producing the crystalline form of AP1189 of Pattern 1, said method comprising:
i. providing an AP1189 salt comprising 2-{3-[1-(2-nitrophenyl)-1H-pyrrol-2-yl]prop-2-en-1-ylidine}hydrazine-1-carboximidamide and a counter ion,
ii. separating the counter ion and 2-{3-[1-(2-nitrophenyl)-1H-pyrrol-2-yl]prop-2-en-1-ylidine}hydrazine-1-carboximidamide using ion exchange, and
iii. isolating the crystalline form of AP1189 of Pattern 1.

Another aspect of the present disclosure provides for pharmaceutical compositions comprising the crystalline form of AP1189.

### DESCRIPTION OF DRAWINGS

FIG. 1: XRPD diffractogram for AP1189 tosylate salt Pattern 1 crystallised from methanol.

FIG. 2: XRPD diffractogram for AP1189 fumarate salt Pattern 1 crystallised from isopropylalcohol:water 90:10 v/v.

FIG. 3: TGA/DSC thermogram of AP1189 tosylate Pattern 1 from IPA:water 90:10 v/v after storage at 40 °C. Peak temperature: 239.24 °C; onset: 233.75 °C; enthalpy (normalised): 99.785 J/g. Weight loss: 0.006 mg; weight percent loss: 0.330%.

FIG. 4: TGA/DSC thermogram of fumarate Pattern 1 from 2-propanol:water 90:10. Peak temperature: 218.27 °C; onset: 214.61 °C; enthalpy (normalised): 68.467 J/g. Weight loss: 0.012 mg; weight percent loss: 0.319%.

FIG. 5: XRPD diffractogram for AP1189 freebase Pattern 1 crystallised from DCM.

FIG. 6: XRPD diffractogram for AP1189 freebase Pattern 1 crystallised from DCM.

FIG. 7: XRPD diffractogram for AP1189 freebase Pattern 3 DMSO solvate.

FIG. 8: XRPD diffractogram for AP1189 freebase Pattern 4 DMF solvate.

FIG. 9: XRPD diffractogram for AP1189 freebase Pattern 5 NMP solvate.

FIG. 10: TGA/DSC thermogram of AP1189 Pattern 1 from DCM. Peak temperature: 185.59 °C; onset: 181.20 °C; enthalpy (normalised): 17.549 J/g. First weight loss: 0.092 mg, first weight percent loss: 1.410%, second weight loss: 0.052 mg, second weight percent loss 0.805%, third weight loss: 0.052 mg, third weight percent loss: 0.804%.

FIG. 11: TGA/DSC thermogram of AP1189 Pattern 3 DMSO solvate. Peak temperature: 106.01 °C; onset: 102.89 °C; enthalpy (normalised): 6.6413 J/g. Weight loss: 0.231 mg; weight percent loss: 2.099%.

FIG. 12: TGA/DSC thermogram of AP1189 Pattern 5 NMP solvate. Peak temperature: 89.90 °C; onset: 82.69 °C; enthalpy (normalised): 3.6210 J/g. Weight loss: 0.058 mg; weight percent loss: 1.069%.

### DETAILED DESCRIPTION

### Definitions

By a “compound of formula I”, “compound I”, and “AP1189” is meant the compound 2-{3-[1-(2-nitrophenyl)-1H-pyrrol-2-yl]prop-2-en-1-ylidine}hydrazine-1-carboximidamide, which has the chemical structure of formula I:

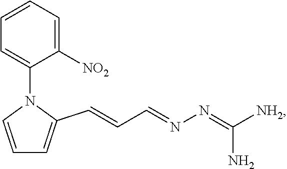 [SMILES: NC(N)=N/N=C/C=C/c1cccn1-c1ccccc1[N+](=O)[O-]]

as well as tautomers and stereoisomers thereof.

As used herein the term “AP1189” refers to the free base structure of Formula I. Preferably, the term “AP1189 free base” refers to the structure of Formula I. By “free base” is meant the compound not forming part of a salt. “AP1189”, “AP1189 freebase”, and “AP1189 free base” are used interchangeably herein.

2-{3-[1-(2-Nitrophenyl)-1H-pyrrol-2-yl]prop-2-en-1-ylidine}hydrazine-1-carboximidamide is also named N-{3-[1-(2-nitrophenyl)-1H-pyrrol-2-yl]-allylidene}-aminoguanidine. “Resomelagon” has been proposed as the international nonproprietary name (INN) for 2-{3-[1-(2-nitrophenyl)-1H-pyrrol-2-yl]prop-2-en-1-ylidine}hydrazine-1-carboximidamide.

Regarding the naming of salts, it is to be construed that terms such as “N-{3-[1-(2-nitrophenyl)-1H-pyrrol-2-yl]-allylidene}-aminoguanidine tosylate”, N-{3-[1-(2-nitrophenyl)-1H-pyrrol-2-yl]-allylidene}-aminoguanidinium tosylate”, and N-{3-[1-(2-nitrophenyl)-1H-pyrrol-2-yl]-allylidene}-aminoguanidine para-toluene sulfonic acid salt are synonymous, i.e. when an anion is written immediately after “N-{3-[1-(2-nitrophenyl)-1H-pyrrol-2-yl]-allylidene}-aminoguanidine”, then the protonated form of “N-{3-[1-(2-nitrophenyl)-1H-pyrrol-2-yl]-allylidene}-aminoguanidine” is meant, i.e. “N-{3-[1-(2-nitrophenyl)-1H-pyrrol-2-yl]-allylidene}-aminoguanidinium”. Similarly, when “N-{3-[1-(2-nitrophenyl)-1H-pyrrol-2-yl]-allylidene}-aminoguanidine” is followed by an acid and “salt”, then the protonated from “N-{3-[1-(2-nitrophenyl)-1H-pyrrol-2-yl]-allylidene}-aminoguanidinium” is meant. These considerations also apply to the name 2-{3-[1-(2-nitrophenyl)-1H-pyrrol-2-yl]prop-2-en-1-ylidine}hydrazine-1-carboximidamide, where the terms “2-{3-[1-(2-nitrophenyl)-1H-pyrrol-2-yl]prop-2-en-1-ylidine}hydrazine-1-carboximidamide tosylate” and “2-{3-[1-(2-nitrophenyl)-1H-pyrrol-2-yl]prop-2-en-1-ylidine}hydrazine-1-carboximidamidium tosylate” have the same meaning herein. These considerations also apply to other salts of the disclosed compound.

In one embodiment, the compound of the disclosure is 2-{3-[1-(2-nitrophenyl)-1H-pyrrol-2-yl]prop-2-en-1-ylidine}hydrazine-1-carboximidamide, including tautomers and stereoisomers thereof.

In one embodiment, the compound of the disclosure is (2E)-2-{3-[1-(2-nitrophenyl)-1H-pyrrol-2-yl]prop-2-en-1-ylidine}hydrazine-1-carboximidamide, including tautomers and stereoisomers thereof.

In one embodiment, the compound of the disclosure is 2-{(2E)-3-[1-(2-nitrophenyl)-1H-pyrrol-2-yl]prop-2-en-1-ylidine}hydrazine-1-carboximidamide, including tautomers and stereoisomers thereof.

In one embodiment, the compound of the disclosure is (2E)-2-{(2E)-3-[1-(2-nitrophenyl)-1H-pyrrol-2-yl]prop-2-en-1-ylidine}hydrazine-1-carboximidamide (also termed (E)-N-trans-{3-[1-(2-nitrophenyl)-1H-pyrrol-2-yl]-allylidene}-aminoguanidine)), including tautomers thereof. These compounds may also appear as the salts and corresponding crystalline forms disclosed herein. In one embodiment, the compound of the disclosure is selected from the group consisting of (2Z)-2-{3-[1-(2-nitrophenyl)-1H-pyrrol-2-yl]prop-2-en-1-ylidine}hydrazine-1-carboximidamide, 2-{(2Z)-3-[1-(2-nitrophenyl)-1H-pyrrol-2-yl]prop-2-en-1-ylidine}hydrazine-1-carboximidamide, (2Z)-2-{(2Z)-3-[1-(2-nitrophenyl)-1H-pyrrol-2-yl]prop-2-en-1-ylidine}hydrazine-1-carboximidamide, (2Z)-2-{(2E)-3-[1-(2-nitrophenyl)-1H-pyrrol-2-yl]prop-2-en-1-ylidine}hydrazine-1-carboximidamide, and (2E)-2-{(2Z)-3-[1-(2-nitrophenyl)-1H-pyrrol-2-yl]prop-2-en-1-ylidine}hydrazine-1-carboximidamide. The compound of the disclosure may additionally be any tautomer of the above structures. As used herein, “tautomer” means other structural isomers that exist in equilibrium resulting from the migration of a hydrogen atom.

In reporting results of a measurement, such as the measurement of a 2-theta value, e.g. the reading of a 2-theta value from an XRPD diffractogram, the skilled person will understand that the method of measuring the value inherently comprises some degree of uncertainty. For example, measurements of 2-theta values may have an uncertainty of 0.2°.

By a “crystalline form of AP1189” is meant the crystalline form of AP1189 (free base) that exhibits the X-ray lines (2-theta values) in a powder diffraction pattern when measured using Cu K_{α} radiation corresponding to AP1189 Pattern 1 as disclosed herein.

Unless otherwise specified, the unit of 2-theta values is degrees (°).

By “onset temperature” is meant the designed intersection point of the extrapolated baseline and the inflectional tangent at the beginning of the melting.

As used herein, “seeding” refers to the technique of adding a “seed” crystal to the crystallization solution to promote the formation of crystals. Preferably, the composition of the seed crystal is the same as the composition of the crystals being formed.

The terms “approximately” and “about” as referred herein are synonymous. In some embodiments, “approximately” and “about” refer to the recited amount, value, or duration ±5%, ±4.5%, ±4%, ±3.5%, ±3%, ±2.5%, ±2%, ±1.75%, ±1.5%, ±1.25%, ±1%, ±0.9%, ±0.8%, ±0.7%, ±0.6%, ±0.5%, ±0.4%, ±0.3%, ±0.2%, ±0.1%, ±0.09%, ±0.08%, ±0.07%, ±0.06%, ±0.05%, ±0.04%, ±0.03%, ±0.02%, or ±0.01%. In some embodiments, “approximately” and “about” refer to the listed amount, value, or duration ±2.5%, ±2%, ±1.75%, ±1.5%, ±1.25%, ±1%, ±0.9%, ±0.8%, ±0.7%, ±0.6%, ±0.5%. In some embodiments, “approximately” and “about” refer to the listed amount, value, or duration ±1%. In some embodiments, “approximately” and “about” refer to the listed amount, value, or duration ±0.5%. In some embodiments, “approximately” and “about” refer to the listed amount, value, or duration ±0.1%.

### Compounds

In one embodiment, the present disclosure provides the compound AP1189. One embodiment provides for the compound AP1189, including tautomeric forms thereof and/or isomeric forms thereof, such as enantiomeric forms and/or diastereomeric forms thereof. In one embodiment, the diastereomeric forms comprise cis and trans forms of the compound, specifically with respect to the alkene moiety. The compound may also exist as either the E or Z form with respect to the C═N double bond of the structure of Formula I. The person of skill in the art understands that in certain instances, E configuration is synonymous to trans configuration, and that in certain instances, Z configuration is synonymous to cis configuration. For example, in the specific case where both of the atoms forming part of a double bond are each bound to exactly 1 further moiety that is not a hydrogen moiety or a lone pair. In one embodiment the term “compound of the disclosure” means the crystalline form of AP1189, that is the crystalline form of AP1189 free base.

### Crystalline Forms

The present disclosure relates to crystalline forms of 2-{3-[1-(2-nitrophenyl)-1H-pyrrol-2-yl]prop-2-en-1-ylidine}hydrazine-1-carboximidamide. It is an object of the disclosure to provide crystalline forms of 2-{3-[1-(2-nitrophenyl)-1H-pyrrol-2-yl]prop-2-en-1-ylidine}hydrazine-1-carboximidamide having high solubility in aqueous medium, over a range of pH values, in particular low pH values.

Crystalline forms of AP1189 and salts thereof may be characterised by X-Ray Powder Diffraction (XRPD) analysis. Such analysis may be carried out using a suitable X-ray powder diffractometer such as a PANalytical X'pert pro with PIXcel detector (128 channels). Scanning of samples may be performed between 3 and 35° 2θ. Samples may be gently ground prior to measurement to release any agglomerates. Samples may be loaded onto a multi-well plate with Kapton or Mylar polymer film to support the sample. Measurements may be carried out by placing the multi-well plate in the diffractometer followed by analysis using Cu K radiation (α1λ=1.54060 Å; α2=1.54443 Å; β=1.39225 Å; α1:α2 ratio=0.5) running in transmission mode (step size 0.0130° 2θ, step time 18.87 s) using 40 kV/40 mA generator settings.

The present disclosure provides for a crystalline form of AP1189. The crystalline form of AP1189 exhibits an XRPD diffractogram as shown in FIG. 5. One embodiment of the present disclosure provides for a crystalline form of AP1189 exhibiting at least X-ray lines (2-theta values) in a powder diffraction pattern when measured using Cu K_{α} radiation at 14.5±0.2, 23.9±0.2, and 26.7±0.2. One embodiment provides for a crystalline form of AP1189, further exhibiting one or more X-ray lines (2-theta values) in a powder diffraction pattern when measured using Cu K_{α} radiation selected from the group consisting of 11.6±0.2, 12.3±0.2, 13.4±0.2, 16.9±0.2, 17.6±0.2, 19.1±0.2, 19.7±0.2, 21.2±0.2, and 27.0±0.2. One embodiment provides for a crystalline form of AP1189 exhibiting an X-ray pattern (2-theta values) in a powder diffraction when measured using Cu K_{α} radiation according to FIG. 5.

One embodiment of the disclosure provides for a crystalline form of AP1189 exhibiting one or more X-ray lines (2 theta values) in a powder diffraction pattern when measured using Cu K_{α} radiation selected from the group consisting of 8.5, 9.8, 11.6, 11.9, 12.3, 12.9, 13.4, 13.6, 14.1, 14.5, 15.1, 16.9, 17.2, 17.6, 18.2, 19.1, 19.4, 19.7, 20.2, 20.6, 21.2, 21.5, 22.0, 22.8, 23.9, 24.8, 25.4, 25.9, 26.3, 26.7, 27.0, 27.4, 27.8, 28.1, 28.5, 28.8, 29.2, 29.4, and 29.8.

One embodiment of the disclosure provides for a crystalline form of AP1189 exhibiting one or more X-ray lines (2 theta values) in a powder diffraction pattern when measured using Cu K_{α} radiation selected from the group consisting of 8.5±0.2, 9.8±0.2, 11.6±0.2, 11.9±0.2, 12.3±0.2, 12.9±0.2, 13.4±0.2, 13.6±0.2, 14.1±0.2, 14.5±0.2, 15.1±0.2, 16.9±0.2, 17.2±0.2, 17.6±0.2, 18.2±0.2, 19.1±0.2, 19.4±0.2, 19.7±0.2, 20.2±0.2, 20.6±0.2, 21.2±0.2, 21.5±0.2, 22.0±0.2, 22.8±0.2, 23.9±0.2, 24.8±0.2, 25.4±0.2, 25.9±0.2, 26.3±0.2, 26.7±0.2, 27.0±0.2, 27.4±0.2, 27.8±0.2, 28.1±0.2, 28.5±0.2, 28.8±0.2, 29.2±0.2, 29.4±0.2, and 29.8±0.2.

It may be advantageous to identify the crystalline form of AP1189 by X-ray lines (2-theta values) having a high relative intensity, and/or by characteristic X-ray lines. Thus, one embodiment of the present disclosure provides for a crystalline form of AP1189 exhibiting one or more X-ray lines (2 theta values) in a powder diffraction pattern when measured using Cu K_{α} radiation selected from the group consisting of 11.6, 12.3, 13.4, 14.5, 16.9, 17.6, 19.1, 19.7, 21.2, 23.9, 26.7, and 27.0.

One embodiment of the present disclosure provides for a crystalline form of AP1189 exhibiting one or more X-ray lines (2 theta values) in a powder diffraction pattern when measured using Cu K_{α} radiation selected from the group consisting of 11.6±0.2, 12.3±0.2, 13.4±0.2, 14.5±0.2, 16.9±0.2, 17.6±0.2, 19.1±0.2, 19.7±0.2, 21.2±0.2, 23.9±0.2, 26.7±0.2, and 27.0±0.2.

One embodiment of the present disclosure provides for a crystalline form of AP1189 exhibiting one or more X-ray lines (2 theta values) in a powder diffraction pattern when measured using Cu K_{α} radiation selected from the group consisting of the 2-theta values listed in Table 3a.

The crystalline forms of AP1189 disclosed herein may appear as solvates. One embodiment of the present disclosure provides for a crystalline form of AP1189 that is a solvate.

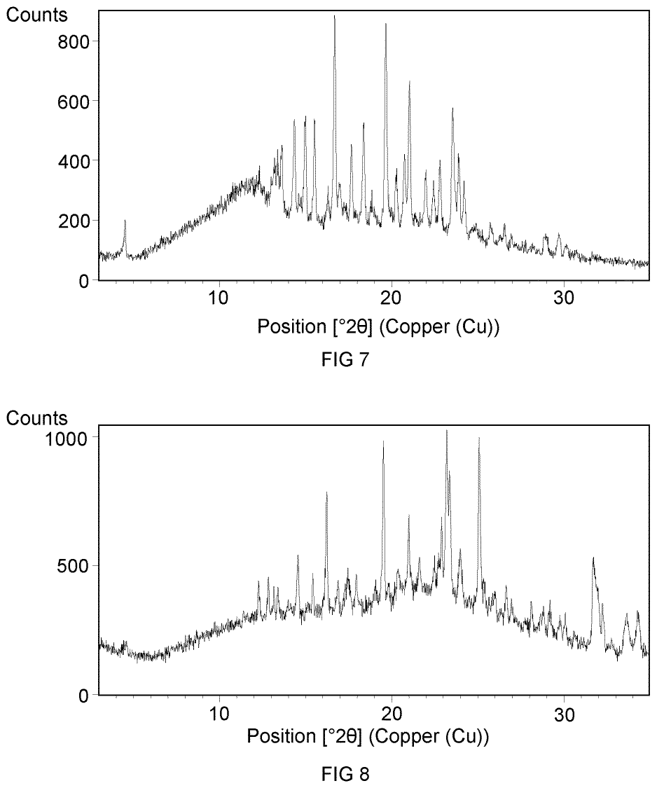

In one embodiment, the crystalline solvate of AP1189 is a DMSO solvate. One embodiment provides for a crystalline form of AP1189 which is a DMSO solvate exhibiting at least X-ray lines (2-theta values) in a powder diffraction pattern when measured using Cu K_{α} radiation at 16.7±0.2, 19.6±0.2, and 21.0±0.2. In a further embodiment, the crystalline form of AP1189 DMSO solvate further exhibits one or more X-ray lines (2-theta values) in a powder diffraction pattern when measured using Cu K_{α} radiation selected from the group consisting of 4.5±0.2, 13.6±0.2, 14.3±0.2, 15.0 0.2, 15.5±0.2, 17.7±0.2, 18.4±0.2, 20.3±0.2, 20.7±0.2, 22.0±0.2, 22.4±0.2, 22.8±0.2, 23.5±0.2, 23.9±0.2, and 24.2±0.2. One embodiment of the disclosure provides for a crystalline form of AP1189 DMSO solvate exhibiting one or more X-ray lines (2 theta values) in a powder diffraction pattern when measured using Cu K_{α} radiation selected from the group consisting of 4.5, 12.3, 13.2, 13.4, 13.6, 14.3, 15.0, 15.5, 16.3, 16.7, 17.0, 17.7, 18.4, 18.8, 19.6, 20.3, 20.7, 21.0, 21.4, 22.0, 22.4, 22.8, 23.5, 23.9, 24.2, 24.8, 25.8, 26.3, 26.6, 27.0, 28.9, 29.7, and 30.1. One embodiment of the disclosure provides for a crystalline form of AP1189 DMSO solvate exhibiting one or more X-ray lines (2 theta values) in a powder diffraction pattern when measured using Cu K_{α} radiation selected from the group consisting of 4.5±0.2, 12.3±0.2, 13.2±0.2, 13.4±0.2, 13.6±0.2, 14.3±0.2, 15.0±0.2, 15.5±0.2, 16.3±0.2, 16.7±0.2, 17.0±0.2, 17.7±0.2, 18.4±0.2, 18.8±0.2, 19.6±0.2, 20.3±0.2, 20.7±0.2, 21.0±0.2, 21.4±0.2, 22.0±0.2, 22.4±0.2, 22.8±0.2, 23.5±0.2, 23.9±0.2, 24.2±0.2, 24.8±0.2, 25.8±0.2, 26.3±0.2, 26.6±0.2, 27.0±0.2, 28.9±0.2, 29.7±0.2, and 30.1±0.2. One embodiment of the present disclosure provides for a crystalline form of AP1189 DMSO solvate exhibiting one or more X-ray lines (2 theta values) in a powder diffraction pattern when measured using Cu K_{α} radiation selected from the group consisting of the 2-theta values listed in Table 3b. One embodiment provides for a crystalline form of AP1189 DMSO solvate exhibiting an X-ray pattern (2-theta values) in a powder diffraction when measured using Cu K_{α} radiation according to FIG. 7.

In one embodiment, the crystalline solvate of AP1189 is a DMF solvate. One embodiment provides for a crystalline form of AP1189 which is a DMF solvate exhibiting at least X-ray lines (2-theta values) in a powder diffraction pattern when measured using Cu K_{α} radiation at 23.2±0.2, 23.4±0.2, and 25.1±0.2. In a further embodiment, the crystalline form of AP1189 DMF solvate further exhibits one or more X-ray lines (2-theta values) in a powder diffraction pattern when measured using Cu K_{α} radiation selected from the group consisting of 12.3±0.2, 12.9±0.2, 14.6±0.2, 15.4±0.2, 16.2±0.2, 16.9±0.2, 17.5±0.2, 17.9±0.2, 20.4±0.2, 21.0±0.2, 21.6±0.2, 22.5±0.2, 22.7±0.2, 22.9±0.2, 24.0±0.2, 25.3±0.2, and 26.7±0.2. One embodiment of the disclosure provides for a crystalline form of AP1189 DMF solvate exhibiting one or more X-ray lines (2 theta values) in a powder diffraction pattern when measured using Cu K_{α} radiation selected from the group consisting of 4.6, 11.4, 12.3, 12.9, 13.4, 14.0, 14.6, 15.1, 15.4, 16.2, 16.9, 17.5, 17.8, 17.9, 19.0, 19.5, 19.8, 20.4, 21.0, 21.6, 22.5, 22.7, 22.9, 23.2, 23.4, 24.0, 25.1, 25.3, 25.7, 26.0, 26.7, 26.9, 28.1, 28.6, 28.8, 29.2, 30.1, 30.7, 31.7, 32.3, 33.7, and 34.3. One embodiment of the disclosure provides for a crystalline form of AP1189 DMF solvate exhibiting one or more X-ray lines (2 theta values) in a powder diffraction pattern when measured using Cu K_{α} radiation selected from the group consisting of 4.6±0.2, 11.4±0.2, 12.3±0.2, 12.9±0.2, 13.4±0.2, 14.0±0.2, 14.6±0.2, 15.1±0.2, 15.4±0.2, 16.2±0.2, 16.9±0.2, 17.5±0.2, 17.8±0.2, 17.9±0.2, 19.0±0.2, 19.5±0.2, 19.8±0.2, 20.4±0.2, 21.0±0.2, 21.6±0.2, 22.5±0.2, 22.7±0.2, 22.9±0.2, 23.2±0.2, 23.4±0.2, 24.0±0.2, 25.1±0.2, 25.3±0.2, 25.7±0.2, 26.0±0.2, 26.7±0.2, 26.9±0.2, 28.1±0.2, 28.6±0.2, 28.8±0.2, 29.2±0.2, 30.1±0.2, 30.7±0.2, 31.7±0.2, 32.3±0.2, 33.7±0.2, and 34.3±0.2. One embodiment of the present disclosure provides for a crystalline form of AP1189 DMF solvate exhibiting one or more X-ray lines (2 theta values) in a powder diffraction pattern when measured using Cu K_{α} radiation selected from the group consisting of the 2-theta values listed in Table 3c. One embodiment provides for a crystalline form of AP1189 DMF solvate exhibiting an X-ray pattern (2-theta values) in a powder diffraction when measured using Cu K_{α} radiation according to FIG. 8.

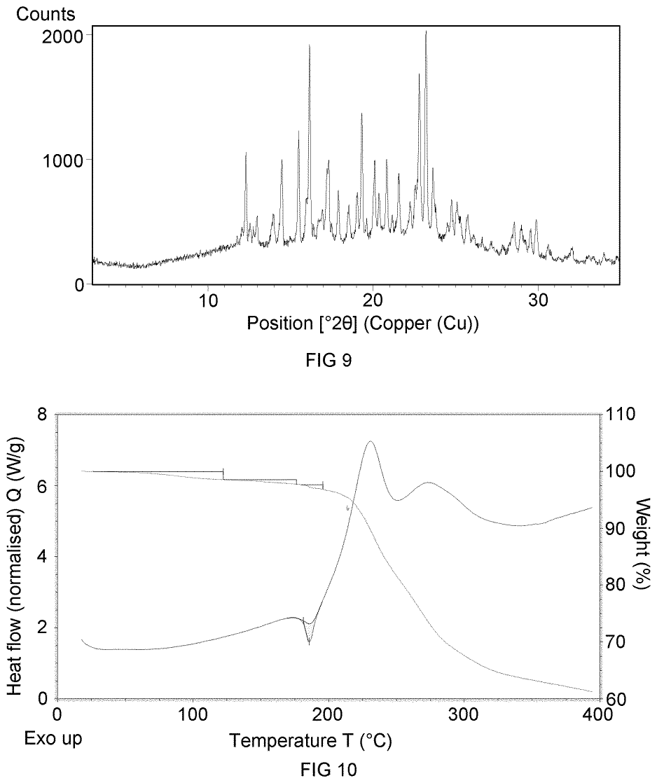

In one embodiment, the crystalline solvate of AP1189 is an NMP solvate. One embodiment provides for a crystalline form of AP1189 which is an NMP solvate exhibiting at least X-ray lines (2-theta values) in a powder diffraction pattern when measured using Cu K_{α} radiation at 16.2±0.2, 22.8±0.2, and 23.2±0.2. In a further embodiment, the crystalline form of AP1189 NMP solvate further exhibits one or more X-ray lines (2-theta values) in a powder diffraction pattern when measured using Cu K_{α} radiation selected from the group consisting of 12.3±0.2, 14.0 0.2, 14.5±0.2, 15.5±0.2, 17.2±0.2, 17.3±0.2, 17.9±0.2, 18.5±0.2, 19.0±0.2, 19.3±0.2, 20.1±0.2, 20.4±0.2, 20.8±0.2, 21.6±0.2, 22.5±0.2, 23.6±0.2, 24.8±0.2, 25.1±0.2, 28.6±0.2, 29.0±0.2, 29.6±0.2, and 29.9±0.2. One embodiment of the disclosure provides for a crystalline form of AP1189 NMP solvate exhibiting one or more X-ray lines (2 theta values) in a powder diffraction pattern when measured using Cu K_{α} radiation selected from the group consisting of 11.7, 12.1, 12.3, 12.6, 12.8, 13.0, 14.0, 14.5, 14.9, 15.5, 16.2, 16.4, 16.7, 16.9, 17.2, 17.3, 17.9, 18.5, 19.0, 19.3, 19.6, 20.1, 20.4, 20.8, 21.2, 21.6, 22.3, 22.5, 22.8, 23.2, 23.6, 24.5, 24.8, 25.1, 25.7, 26.1, 26.6, 27.1, 27.8, 28.3, 28.6, 29.0, 29.6, 29.9, 30.6, 32.1, 33.2, and 34.1. One embodiment of the disclosure provides for a crystalline form of AP1189 NMP solvate exhibiting one or more X-ray lines (2 theta values) in a powder diffraction pattern when measured using Cu K_{α} radiation selected from the group consisting of 11.7±0.2, 12.1±0.2, 12.3±0.2, 12.6±0.2, 12.8±0.2, 13.0±0.2, 14.0±0.2, 14.5±0.2, 14.9±0.2, 15.5±0.2, 16.2±0.2, 16.4±0.2, 16.7±0.2, 16.9±0.2, 17.2±0.2, 17.3±0.2, 17.9±0.2, 18.5±0.2, 19.0±0.2, 19.3±0.2, 19.6±0.2, 20.1±0.2, 20.4±0.2, 20.8±0.2, 21.2±0.2, 21.6±0.2, 22.3±0.2, 22.5±0.2, 22.8±0.2, 23.2±0.2, 23.6±0.2, 24.5±0.2, 24.8±0.2, 25.1±0.2, 25.7±0.2, 26.1±0.2, 26.6±0.2, 27.1±0.2, 27.8±0.2, 28.3±0.2, 28.6±0.2, 29.0±0.2, 29.6±0.2, 29.9±0.2, 30.6±0.2, 32.1±0.2, 33.2±0.2, and 34.1±0.2. One embodiment of the present disclosure provides for a crystalline form of AP1189 NMP solvate exhibiting one or more X-ray lines (2 theta values) in a powder diffraction pattern when measured using Cu K_{α} radiation selected from the group consisting of the 2-theta values listed in Table 3d. One embodiment provides for a crystalline form of AP1189 NMP solvate exhibiting an X-ray pattern (2-theta values) in a powder diffraction when measured using Cu K_{α} radiation according to FIG. 9.

One embodiment of the present disclosure provides for a polymorph of AP1189 freebase.

### Further Characterisation of Crystalline Forms

The crystalline form of AP1189 provided herein may be further characterised by the onset temperatures it exhibits as assessed by differential scanning calorimetry.

One embodiment of the present disclosure provides for a crystalline form of AP1189 exhibiting in differential scanning calorimetry an onset temperature between 174 and 188 °C. One specific embodiment of the present disclosure provides a crystalline form of AP1189 exhibiting in differential scanning calorimetry an onset temperature of substantially 181 °C. In a further embodiment, the onset temperature is assessed using a heating rate of 10 °C/min. One embodiment of the disclosure provides for a crystalline form of AP1189 exhibiting in differential scanning calorimetry an onset temperature as shown in the examples herein, specifically example 4, and/or in the figures herein, specifically FIG. 10. One embodiment of the present disclosure provides a crystalline form of AP1189 exhibiting a differential scanning calorimetry thermogram according to FIG. 10. One embodiment of the present disclosure provides for a crystalline form of AP1189 exhibiting in differential scanning calorimetry an onset temperature falling within the interval 181±7 °C, such as 181±6 °C, such as 181±5 °C, such as 181±4 °C, such as 181±3 °C, such as 181±2 °C, such as 181±1 °C.

One embodiment of the present disclosure provides for a crystalline form of AP1189 DMSO solvate exhibiting in differential scanning calorimetry an onset temperature between 96 and 110 °C. One specific embodiment of the present disclosure provides a crystalline form of AP1189 DMSO solvate exhibiting in differential scanning calorimetry an onset temperature of substantially 103 °C. In a further embodiment, the onset temperature is assessed using a heating rate of 10 °C/min. One embodiment of the disclosure provides for a crystalline form of AP1189 DMSO solvate exhibiting in differential scanning calorimetry an onset temperature as shown in the examples herein, specifically example 4, and/or in the figures herein, specifically FIG. 11. One embodiment of the present disclosure provides a crystalline form of AP1189 DMSO solvate exhibiting a differential scanning calorimetry thermogram according to FIG. 11. One embodiment of the present disclosure provides for a crystalline form of AP1189 DMSO solvate exhibiting in differential scanning calorimetry an onset temperature falling within the interval 103±7 °C, such as 103±6 °C, such as 103±5 °C, such as 103±4 °C, such as 103±3 °C, such as 103±2 °C, such as 103±1 °C.

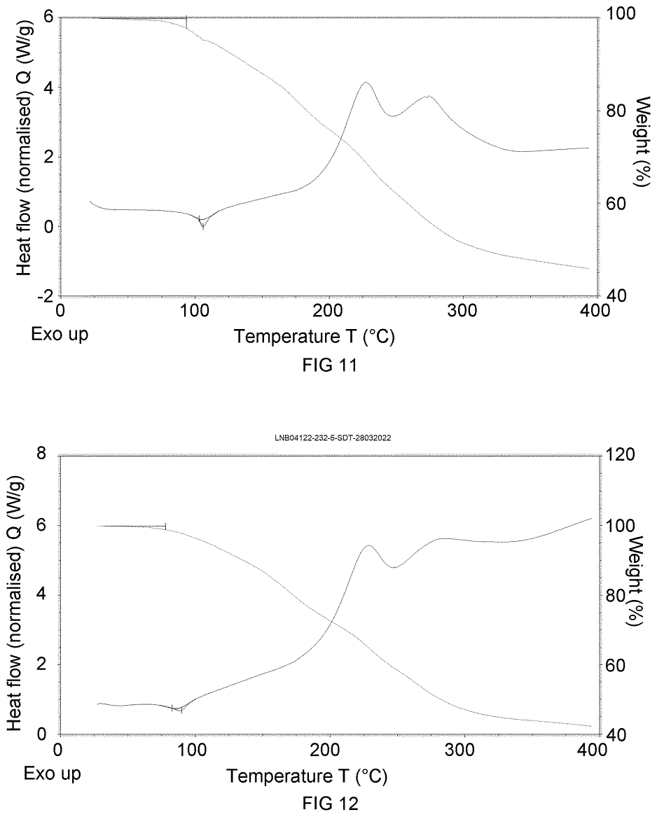

One embodiment of the present disclosure provides for a crystalline form of AP1189 NMP solvate exhibiting in differential scanning calorimetry an onset temperature between 76 and 90 °C. One specific embodiment of the present disclosure provides a crystalline form of AP1189 NMP solvate exhibiting in differential scanning calorimetry an onset temperature of substantially 83 °C. In a further embodiment, the onset temperature is assessed using a heating rate of 10 °C/min. One embodiment of the disclosure provides for a crystalline form of AP1189 NMP solvate exhibiting in differential scanning calorimetry an onset temperature as shown in the examples herein, specifically example 4, and/or in the figures herein, specifically FIG. 12. One embodiment of the present disclosure provides a crystalline form of AP1189 NMP solvate exhibiting a differential scanning calorimetry thermogram according to FIG. 12. One embodiment of the present disclosure provides for a crystalline form of AP1189 NMP solvate exhibiting in differential scanning calorimetry an onset temperature falling within the interval 83±7 °C, such as 83±6 °C, such as 83±5 °C, such as 83±4 °C, such as 83±3 °C, such as 83±2 °C, such as 83±1 °C.

### Properties of Crystalline Forms

The present disclosure provides a polymorph of AP1189 (AP1189 freebase) having high solubility in acidic and slightly acidic conditions. Specifically, as shown in the examples herein, the crystalline form of AP1189 free base was found to have a high solubility at pH 1.2 compared to the test compound AP1189 fumarate Pattern 1 (see table 8). AP1189 freebase Pattern 1 also exhibited a high solubility at pH 4.5 compared to test compound AP1189 fumarate Pattern 1.

It is an object of the present disclosure to provide forms of AP1189 having a high solubility at low pH (acidic pH, acidic conditions; about pH 1.2), as this can improve in vivo uptake of AP1189 after administration to a subject, such as oral administration to a subject.

It is also an object of the present disclosure to provide forms of AP1189 having a high solubility at slightly acidic conditions (about pH 4.5), as this can improve in vivo uptake of AP1189 after administration to a subject, such as oral administration to a subject.

High solubility of compounds derived from AP1189, specifically AP1189 salts, at acidic conditions, is not a given, as is shown in the examples herein. For example, both A P1189 tosylate and AP1189 fumarate were found to have low solubility at low pH, e.g. pH 1.2-1.3 or pH 4.5.

One embodiment of the present disclosure provides for a crystalline form of AP1189 having a solubility at pH 1.2 of at least 15 mM, such as at least 20 mM, such as at least 25 mM, such as at least 30 mM, such as at least 35 mM, such as at least 40 mM. One embodiment of the present disclosure provides for a crystalline form of AP1189 having a solubility at pH 4.5 of at least 10 mM, such as at least 15 mM, such as at least 20 mM.

The solubility of a compound may be assessed by adding a surplus of the compound to a volume of solvent such that some of the compound is not dissolved, then isolating the non-dissolved compound and measuring the amount. The solubility of a compound may alternatively be assessed by adding a surplus of the compound to a volume of solvent such that some of the compound is not dissolved, and then measure the amount of compound in solution. Measuring the amount of compound in solution may be done using any suitable method, such as HPLC, titration, or spectrometry.

### Methods for Preparing Crystalline Forms of AP1189

Crystalline forms of AP1189 may be prepared as disclosed herein.

One embodiment of the present disclosure provides for a method for producing a crystalline form of AP1189 of Pattern 1, said method comprising:
i. mixing AP1189 and a solvent to form a mixture; and
ii. isolating the crystalline form of AP1189 of Pattern 1 from said mixture.

As used herein, “mixture” can mean a solution or a slurry of one or more solids in a solvent or mixture of solvents. In one embodiment of the disclosure, the mixture is a solution, where the solute or solutes are substantially fully dissolved. In one embodiment, the mixture is a slurry, wherein one or more solutes are only partly dissolved, and the remaining part or parts of the solute or solutes are not dissolved.

The AP1189 freebase of Pattern 1 as disclosed herein may be obtained from an AP1189 salt using a so-called “salt break” to produce the AP1189 freebase. One embodiment of the present disclosure provides for a method for producing the crystalline form of AP1189 of Pattern 1, said method comprising:
i. mixing an AP1189 salt and a solvent to form a composition; and
ii. isolating the crystalline form of AP1189 of Pattern 1 from said composition.

As used herein, “composition” can mean a solution or a slurry of one solid in a solvent or mixture of solvents. In one embodiment of the disclosure, the composition is a solution, where the solute is substantially fully dissolved. In one embodiment, the composition is a slurry, wherein the solute is only partly dissolved, and the remaining part of the solute is not dissolved. The composition may further comprise one or more other agents or reagents which may be dissolved or may be only partly dissolved. Such other agents includes, but are not limited to, surfactants, detergents, acids, bases, sugars, salts, biomolecules, bioactive agents, and other excipients such as pharmaceutical excipients.

This present disclosure also relates to non-solid compositions, e.g. liquid compositions, gel compositions, pastes, creams, or ointments prepared from the crystalline forms disclosed herein. One embodiment provides for a liquid composition, gel composition, paste, cream, or ointment prepared from a crystalline form disclosed herein. One specific embodiment provides for a liquid composition prepared from a crystalline form disclosed herein and a solvent. In a specific embodiment, the solvent is aqueous. In one embodiment, the present disclosure provides for a method of preparing a liquid composition, a gel composition, a paste, a cream, or an ointment, said method comprising mixing a crystalline form disclosed herein and one or more additional agents. One specific embodiment provides for a method of preparing a liquid composition, said method comprising mixing a crystalline form disclosed herein and a solvent. In a further embodiment, the solvent is aqueous.

The crystalline form of AP1189 of Pattern 1 may be obtained directly from a reaction producing AP1189 from one or more precursors. AP1189 may be produced from a condensation reaction of 3-[1-(2-nitrophenyl)-1-H-pyrrole-2-yl]-propanal and amino guanidine. Thus, one embodiment of the present disclosure provides for a method for producing the crystalline form of AP1189 of Pattern 1, said method comprising:
i. mixing 3-[1-(2-nitrophenyl)-1-H-pyrrole-2-yl]-propanal and amino guanidine or a salt thereof in a solvent to form a composition, and
ii. isolating the crystalline form of AP1189 of Pattern 1 from said composition.

Any one of the above reagents of step i may be generated from a precursor in situ.

The crystalline form of AP1189 of Pattern may be obtained from a salt break of an AP1189 salt using ion exchange. Thus, one embodiment of the present disclosure provides for a method for producing the crystalline form of AP1189 of Pattern 1, said method comprising:
i. providing an AP1189 salt comprising 2-{3-[1-(2-nitrophenyl)-1H-pyrrol-2-yl]prop-2-en-1-ylidine}hydrazine-1-carboximidamide and a counter ion,
ii. separating the counter ion and 2-{3-[1-(2-nitrophenyl)-1H-pyrrol-2-yl]prop-2-en-1-ylidine}hydrazine-1-carboximidamide using ion exchange, and
iii. isolating the crystalline form of AP1189 of Pattern 1.

In one embodiment of the present disclosure, the solvent is a protic or a polar aprotic solvent. In one embodiment, the solvent is selected from the group consisting of 2-methyl tetrahydrofuran, 2-propanol, ethanol, water, acetone, acetonitrile, dichloromethane, dimethylsulfoxide, ethyl acetate, heptane, methanol, methylethyl ketone, methylisobutyl ketone, tert-butylmethyl ether, tetrahydrofuran, dimethylformamide (DMF), N-methyl-2-pyrrolidone (NMP), and toluene. In one embodiment, the solvent is a mixture of two or more solvents, such as a mixture of 2-propanol and water, or a mixture of ethanol and water.

In one embodiment of the present disclosure, the mixture or the composition is heated at least once before the isolating step. In one embodiment, the mixture or the composition is heated and cooled in cycles before the isolation step. In one embodiment, the mixture or the composition is heated and cooled in cycles for up to 72 hours before the isolating step. In one embodiment the mixture or the composition is heated and cooled in cycles for 15 min to 72 hours before the isolating step. In one embodiment, one cycle comprises heating the mixture or the composition to at least a first threshold temperature, maintaining the temperature above said first threshold temperature for a first duration, then cooling the mixture or the composition to below a second threshold temperature and maintaining the temperature below said second threshold temperature for a second duration. In one embodiment of the present disclosure, the cycle is carried out 1 to 200 times. In one embodiment of the present disclosure, the first threshold temperature is 30 °C, such as 35 °C, such as 40 °C, such as 45 °C, such as 50 °C, such as 55 °C, such as 60 °C, such as 65 °C, such as 70 °C, such as 75 °C, such as 80 °C. In one embodiment of the present disclosure, the second threshold temperature is 30 °C, such as 25 °C, such as 20 °C, such as 15 °C, such as 10 °C, such as 7 °C, such as 5 °C. In one embodiment of the present disclosure, the first and/or the second duration is 1 to 2 min, such as 2 to 5 min, such as 5 to 10 min, such as 10 to 20 min, such as 20 to 30 min, such as 30 to 40 min, such as 40 to 50 min, such as 50 to 60 min, such as 1 hour to 1.5 hours, such as 1.5 to 2 hours, such as 2 to 3 hours, such as 3 to 4 hours, such as 4 to 5 hours, such as 5 to 6 hours, such as 6 to 7 hours, such as 7 to 8 hours. In one embodiment, the first and the second durations are the same. In one embodiment, the first and the second durations are different. In one embodiment, the first duration is different or the same for each cycle. In one embodiment, the second duration is different or the same for each cycle.

In one embodiment, heating is to about 40 °C.

In one embodiment, cooling is to about 20 °C.

In one embodiment, the method further comprises a step of adding an anti-solvent to the mixture or the composition before the isolation step. In one embodiment, the anti-solvent is a non-polar aprotic solvent. In one embodiment, the anti-solvent is selected from the group consisting of tert-butyl methyl ether, THF, and acetone, and mixtures comprising tert-butyl methyl ether, THF, or acetone. In one embodiment of the present disclosure, the anti-solvent is water. In one embodiment, the anti-solvent is a mixture of two or more solvents.

Isolation of crystals may be carried out using any appropriate means. In one embodiment of the disclosure, the isolation is carried out using filtration, centrifugation, and/or evaporation of the solvent or solvents. In one embodiment, a slow evaporation method is utilised. In one embodiment, a fast evaporation method is utilised. In one embodiment, the evaporation is carried out using spray drying. In one embodiment, the evaporation is carried out using fluid bed drying, freeze drying, vacuum drying, tumble drying, rotary evaporation, and/or thin-film evaporation. In one embodiment, the drying is carried out using a conductive (contact) dryer, including tray dryers, rotary cone dryers, tumble dryers, and paddle dryers. In one further embodiment, the drying is carried out using a carrier gas.

One embodiment provides for a method as disclosed here, further comprising the step of adding a seed crystal of the crystalline form of AP1189 of Pattern 1.

One embodiment provides for a crystalline form of AP1189 of Pattern 1 produced by a method disclosed herein.

One embodiment provides for a method of preparing an amorphous form of AP1189, said method comprising converting the crystalline form of AP1189 of Pattern 1 to the amorphous form of AP1189.

Pharmaceutical Compositions (Crystalline Form) One embodiment of the disclosure provides for a pharmaceutical composition comprising the crystalline form of AP1189 free base as disclosed herein.

One embodiment of the disclosure provides for a pharmaceutical composition comprising the crystalline form of AP1189 free base as disclosed herein and a pharmaceutically acceptable excipient.

In some embodiments there is provided an oral formulation, a pharmaceutical composition, or unit dosage form comprising the crystalline form a of AP1189 free base as disclosed herein.

One embodiment provides for a pharmaceutical composition as disclosed herein, wherein the pharmaceutical composition is formulated for oral administration. Such composition may be in the form of a tablet or a capsule.

One embodiment of the disclosure provides for a method of preparing a pharmaceutical composition comprising mixing the crystalline form of AP1189 free base with a pharmaceutically acceptable excipient.

One embodiment of the disclosure provides for the crystalline form of AP1189 free base or the pharmaceutical composition as disclosed herein for use in medicine. One embodiment of the present disclosure provides for the crystalline form of AP1189 free base or the pharmaceutical composition as disclosed herein, for use in the treatment of a kidney disease such as proteinuria, a cardiovascular disease, an arthritic disease, or a viral infection.

One embodiment of the present disclosure provides for a method of treating a disease or disorder in a subject in need thereof, said method comprising administering crystalline form of AP1189 free base or the pharmaceutical composition as disclosed herein, to a subject in need thereof. In one further embodiment, the disease or disorder is selected from the list consisting of a kidney disease such as proteinuria, a cardiovascular disease, an arthritic disease, or a viral infection.

One embodiment of the present disclosure provides for a use of the crystalline form of AP1189 free base or the pharmaceutical composition as disclosed herein for the manufacture of a medicament for treatment of a disease or disorder.

### Pharmaceutical Compositions (Gastric Release)

It is an aspect of the present disclosure to provide a pharmaceutical composition comprising 2-{3-[1-(2-nitrophenyl)-1H-pyrrol-2-yl]prop-2-en-1-ylidine}hydrazine-1-carboximidamide (AP1189 free base), including tautomeric and stereoisomeric forms thereof, having high solubility at low pH.

Reference to a pharmaceutical composition comprising 2-{3-[1-(2-nitrophenyl)-1H-pyrrol-2-yl]prop-2-en-1-ylidine}hydrazine-1-carboximidamide is meant to encompass any forms thereof, including the crystalline form and an amorphous form of 2-{3-[1-(2-nitrophenyl)-1H-pyrrol-2-yl]prop-2-en-1-ylidine}hydrazine-1-carboximidamide.

Some embodiments of the present disclosure provide a pharmaceutical composition comprising the crystalline form of 2-{3-[1-(2-nitrophenyl)-1H-pyrrol-2-yl]prop-2-en-1-ylidine}hydrazine-1-carboximidamide.

Some embodiments of the present disclosure provide a pharmaceutical composition comprising an amorphous form of 2-{3-[1-(2-nitrophenyl)-1H-pyrrol-2-yl]prop-2-en-1-ylidine}hydrazine-1-carboximidamide.

Some embodiments of the present disclosure provide a unit dosage form of a pharmaceutical composition comprising 2-{3-[1-(2-nitrophenyl)-1H-pyrrol-2-yl]prop-2-en-1-ylidine}hydrazine-1-carboximidamide.

Some embodiments of the present disclosure provide an oral formulation comprising 2-{3-[1-(2-nitrophenyl)-1H-pyrrol-2-yl]prop-2-en-1-ylidine}hydrazine-1-carboximidamide.

Some embodiments of the present disclosure provide an oral formulation comprising 2-{3-[1-(2-nitrophenyl)-1H-pyrrol-2-yl]prop-2-en-1-ylidine}hydrazine-1-carboximidamide, and at least one pharmaceutically acceptable excipient.

Some embodiments of the present disclosure provide an oral formulation comprising AP1189 (free base), wherein said AP1189 has a solubility of at least 15 mM, such as at least 20 mM, such as at least 25 mM, such as at least 30 mM, such as at least 35 mM such as at least 40 mM at pH 1.2; such as about pH 1.2.

Some embodiments of the present disclosure provide an oral formulation comprising AP1189 (free base), wherein said AP1189 has a solubility of at least 10 mM, such as at least 15 mM, such as at least 20 mM at pH 4.5; such as about pH 4.5.

In some embodiments said oral formulation delivers said AP1189 free base to the gastric compartment (or stomach). In some embodiments said oral formulation primarily or predominantly delivers said AP1189 free base to the gastric compartment (or stomach).

In some embodiments said oral formulation releases said AP1189 free base in the gastric compartment. In some embodiments said oral formulation primarily or predominantly releases said AP1189 free base in the gastric compartment.

In some embodiments said oral formulation releases said AP1189 free base in the gastric compartment by immediate release, by delayed release, by burst release, or by any means of releasing said AP1189 free base primarily or predominantly in the gastric compartment.

In some embodiments said oral formulation delivers and/or releases not less than about 65% to about 80% of the AP1189 free base in the gastric compartment.

In some embodiments said oral formulation delivers and/or releases not less than about 65% of the AP1189 free base, such as not less than about 70%, such as not less than about 75%, such as not less than about 80%, such as not less than about 85%, such as not less than about 90%, such as not less than about 95% of the AP1189 free base in the gastric compartment.

In some embodiments said oral formulation immediately releases said AP1189 free base in the gastric compartment.

In some embodiments said oral formulation releases said AP1189 free base in the gastric compartment for gastric absorption of said AP1189.

In some embodiments said oral formulation releases said AP1189 free base in the gastric compartment for absorption of said AP1189 over the gastric mucus layer.

In some embodiments said oral formulation is designed for gastric delivery.

In some embodiments said oral formulation is designed for gastric release.

In some embodiments said oral formulation is designed for gastric absorption.

In some embodiments said oral formulation is a solid oral formulation.

It is an aspect of the present disclosure to provide a solid oral formulation comprising 2-{3-[1-(2-nitrophenyl)-1H-pyrrol-2-yl]prop-2-en-1-ylidine}hydrazine-1-carboximidamide, including tautomeric and stereoisomeric forms thereof. Some embodiments of the present disclosure provide a solid oral formulation comprising 2-{3-[1-(2-nitrophenyl)-1H-pyrrol-2-yl]prop-2-en-1-ylidine}hydrazine-1-carboximidamide.

Some embodiments of the present disclosure provide a solid oral formulation comprising 2-{3-[1-(2-nitrophenyl)-1H-pyrrol-2-yl]prop-2-en-1-ylidine}hydrazine-1-carboximidamide, and at least one pharmaceutically acceptable excipient.

It is an aspect of the present disclosure to provide a solid oral dosage form comprising 2-{3-[1-(2-nitrophenyl)-1H-pyrrol-2-yl]prop-2-en-1-ylidine}hydrazine-1-carboximidamide, including tautomeric and stereoisomeric forms thereof. Some embodiments of the present disclosure provide a solid oral dosage form comprising 2-{3-[1-(2-nitrophenyl)-1H-pyrrol-2-yl]prop-2-en-1-ylidine}hydrazine-1-carboximidamide.

Some embodiments of the present disclosure provide a solid oral dosage form comprising 2-{3-[1-(2-nitrophenyl)-1H-pyrrol-2-yl]prop-2-en-1-ylidine}hydrazine-1-carboximidamide, and at least one pharmaceutically acceptable excipient.

In some embodiments said oral formulation is a tablet. In some embodiments said solid oral formulation is a tablet. In some embodiments said solid oral dosage form is a tablet.

Some embodiments of the present disclosure provide a tablet comprising 2-{3-[1-(2-nitrophenyl)-1H-pyrrol-2-yl]prop-2-en-1-ylidine}hydrazine-1-carboximidamide, and at least one pharmaceutically acceptable excipient.

In some embodiments there is provided an oral formulation comprising AP1189 free base, wherein not less than about 65% to about 80% of the AP1189 free base is dissolved into solution in the gastric compartment.

In some embodiments there is provided a solid oral formulation, solid oral dosage form or tablet, comprising AP1189 free base, wherein not less than about 65% to about 80% of the AP1189 free base is dissolved into solution in the gastric compartment.

In some embodiments there is provided a solid oral formulation, solid oral dosage form or tablet, comprising AP1189 free base, wherein not less than about 65%, such as not less than about 70%, such as not less than about 75%, such as not less than about 80%, such as not less than about 85%, such as not less than about 90%, such as not less than about 95% of the AP1189 free base is dissolved into solution in the gastric compartment.

In some embodiments said oral formulation is a delayed release tablet comprising AP1189 free base targeting release in the gastric compartment.

In some embodiments said solid oral formulation is a delayed release solid oral formulation comprising AP1189 free base targeting release in the gastric compartment.

In some embodiments said oral formulation is a gastric retentive delayed-release formulation. In some embodiments said oral formulation is a gastroretentive tablet. In some embodiments said gastroretentive tablet is a gastroretentive double-layered tablet formulation. In some embodiments said gastroretentive double-layered tablet formulation is a gastric swelling system (GSS), such as a GSS consisting of a swelling layer and a drug release layer.

It is an aspect of the present disclosure to provide a gastric retentive delayed release solid oral formulation, solid oral dosage form or tablet, comprising 2-{3-[1-(2-nitrophenyl)-1H-pyrrol-2-yl]prop-2-en-1-ylidine}hydrazine-1-carboximidamide, including tautomeric and stereoisomeric forms thereof.

In some embodiments said oral formulation is an immediate release oral formulation. In some embodiments said solid oral formulation is an immediate release solid oral formulation. In some embodiments said solid oral formulation is an immediate release tablet.

In some embodiments said solid oral formulation is an immediate release tablet comprising AP1189 free base targeting release in the gastric compartment.

It is an aspect of the present disclosure to provide an immediate release solid oral formulation, such as an immediate release solid oral dosage form, such as an immediate release tablet, comprising 2-{3-[1-(2-nitrophenyl)-1H-pyrrol-2-yl]prop-2-en-1-ylidine}hydrazine-1-carboximidamide, including tautomeric and stereoisomeric forms thereof.

In some embodiments there is provided an immediate release solid oral formulation, such as an immediate release solid oral dosage form, such as an immediate release tablet, comprising 2-{3-[1-(2-nitrophenyl)-1H-pyrrol-2-yl]prop-2-en-1-ylidine}hydrazine-1-carboximidamide, including tautomeric and stereoisomeric forms thereof, and at least one pharmaceutically acceptable excipient.

In some embodiments there is provided an oral formulation, such as a solid oral formulation, such as an immediate-release solid oral formulation, comprising AP1189 free base, wherein not less than about 65% to about 80% of the AP1189 free base is dissolved into solution in about 5 minutes.

In some embodiments there is provided an oral formulation, such as a solid oral formulation, such as an immediate-release solid oral formulation, comprising AP1189 free base, wherein not less than about 65 to about 80% of the AP1189 free base is dissolved into solution in about 5 minutes at a pH of about 1 to 3, such as a pH of about 1.5 to 3.5; such as a pH of about 1 to 1.5, such as a pH of about 1.5 to 2, such as a pH of about 2 to 2.5, such as a pH of about 2.5 to 3, such as a pH of about 3 to 3.5.

In some embodiments there is provided an oral formulation, such as a solid oral formulation, such as an immediate-release solid oral formulation, comprising AP1189 free base, wherein not less than about 65 to about 80% of the AP1189 free base is dissolved into solution in about 5 minutes at a pH of about 1.2.

In some embodiments there is provided an oral formulation, such as a solid oral formulation, such as an immediate-release solid oral formulation, comprising AP1189 free base, wherein not less than about 80% of the AP1189 free base is dissolved into solution in about 10 minutes, such as about 15 minutes.

In some embodiments there is provided an oral formulation, such as a solid oral formulation, such as an immediate-release solid oral formulation, comprising AP1189 free base, wherein not less than about 80% of the AP1189 free base is dissolved into solution in about 10 minutes at a pH of about 1 to 3, such as a pH of about 1.5 to 3.5; such as a pH of about 1 to 1.5, such as a pH of about 1.5 to 2, such as a pH of about 2 to 2.5, such as a pH of about 2.5 to 3, such as a pH of about 3 to 3.5.

In some embodiments there is provided an oral formulation, such as a solid oral formulation, such as an immediate-release solid oral formulation, comprising AP1189 free base, wherein not less than about 80% of the AP1189 free base is dissolved into solution in about 10 minutes at a pH of about 1.2, such as in about 15 minutes at a pH of about 1.2.

In some embodiments there is provided an oral formulation, such as a solid oral formulation, such as an immediate-release solid oral formulation, comprising AP1189 free base, wherein the disintegration time of said solid oral formulation is from % minute to 10 minutes, such as % minute to 1 minute, such as 1 to 2 minutes, such as 2 to 3 minutes, such as 3 to 4 minutes, such as 4 to 5 minutes, such as 5 to 6 minutes, such as 6 to 7 minutes, such as 7 to 8 minutes, such as 8 to 9 minutes, such as 9 to 10 minutes.

In some embodiments the solid oral dosage form comprises AP1189 free base at a dosage from about 25 mg to about 650 mg per tablet, such as about 25 mg AP1189 free base, such as about 50 mg, such as about 100 mg, such as about 150 mg, such as about 200 mg, such as about 250 mg, such as about 300 mg, such as about 350 mg, such as about 400 mg, such as about 450 mg, such as about 500 mg, such as about 550 mg, such as about 600 mg, such as about 650 mg AP1189 free base.

In some embodiments the solid oral dosage form comprises AP1189 free base at a dosage of about 25 mg per tablet, such as about 50 mg, such as about 100 mg, such as about 150 mg, such as about 200 mg per tablet.

The solid oral formulation of the present disclosure may be formulated by any means know to the skilled person to provide release of said AP1189 free base in the gastric compartment.

### Dosage

According to the present disclosure, the oral formulation comprising a compound of the disclosure is administered to individuals in need of treatment in pharmaceutically effective doses. A therapeutically effective amount of a compound is an amount sufficient to cure, prevent, reduce the risk of, alleviate or partially arrest the clinical manifestations of a given disease and its complications. The amount that is effective for a particular therapeutic purpose will depend on the severity and the sort of the disorder as well as on the weight and general state of the subject. The compound may be administered one or several times per day, such as from 1 to 8 times per day, such as from 1 to 6 times per day, such as from 1 to 5 times per day, such as from 1 to 4 times per day, such as from 1 to 3 times per day, such as from 1 to 2 times per day, such as from 2 to 4 times per day, such as from 2 to 3 times per day. Alternatively, the compound may be administered less than once a day, for example once a day, such as once every second day, for example once every third day, such as once every fourth day, for example once every fifth day, such as once every sixth day, for example once every week.

In some embodiments the oral formulation comprising a compound of the disclosure is administered in a therapeutically effective amount, such as in an amount of 1 mg to 1000 mg AP1189 free base per day.

It follows that in some embodiments AP1189 free base is administered in an amount of 1 mg to 1000 mg, such as 1 to 5 mg, 5 to 10 mg, 10 to 15 mg, 15 to 20 mg, 20 to 25 mg, 25 to 50 mg, 50 to 75 mg, 75 to 100 mg, 100 to 125 mg, 125 to 150 mg, 150 to 175 mg, 175 to 200 mg, 200 to 250 mg, 250 to 300 mg, 300 to 350 mg, 350 to 400 mg, 400 to 450 mg, 450 to 500 mg, 500 to 600 mg, 600 to 700 mg, 700 to 800 mg, 800 to 900 mg, or 900 to 1000 mg per day.

In some embodiments, ‘per day’ means the dosage is given in one dosage or is divided in multiple dosages per day, including once a day (QD), twice a day (BID) and/or three times a day (TID).

In some embodiments the oral formulation of the present disclosure is administered once daily.

In some embodiments AP1189 free base is administered in an amount of 1 mg to 1000 mg, such as 1 to 5 mg, 5 to 10 mg, 10 to 15 mg, 15 to 20 mg, 20 to 25 mg, 25 to 50 mg, 50 to 75 mg, 75 to 100 mg, 100 to 125 mg, 125 to 150 mg, 150 to 175 mg, 175 to 200 mg, 200 to 250 mg, 250 to 300 mg, 300 to 350 mg, 350 to 400 mg, 400 to 450 mg, 450 to 500 mg, 500 to 600 mg, 600 to 700 mg, 700 to 800 mg, 800 to 900 mg, or 900 to 1000 mg AP1189 per dosage.

In some embodiments AP1189 free base is administered in an amount of 25 mg once daily, 50 mg once daily, 100 mg once daily, 200 mg once daily, 300 mg once daily, 400 mg once daily, 500 mg once daily, 600 mg once daily, 700 mg once daily, 800 mg once daily, 900 mg once daily or 1000 mg once daily.

In some embodiments AP1189 free base is administered in an amount of 50 mg once daily, 50 mg two times daily (BID) or 50 mg three times daily (TID).

In some embodiments AP1189 free base is administered in an amount of 100 mg once daily, 100 mg two times daily (BID) or 100 mg three times daily (TID).

In some embodiments AP1189 free base is administered in an amount of 200 mg once daily, 200 mg two times daily (BID) or 200 mg three times daily (TID).

In some embodiments AP1189 free base is administered in an amount of 400 mg once daily, 400 mg two times daily (BID) or 200 mg three times daily (TID).

In some embodiments a daily dosage of AP1189 free base is split into multiple dosage forms and/or administrations.

In another embodiment AP1189 free base is administered in an amount of 0.1 to 0.5 mg/kg bodyweight, such as 0.5 mg to 1 mg/kg bodyweight, such as 1 to 2 mg/kg bodyweight, such as 2 to 3 mg/kg bodyweight, such as 3 to 5 mg/kg bodyweight, such as 5 to 10 mg/kg bodyweight, such as 10 to 15 mg/kg bodyweight, such as 15 to 20 mg/kg bodyweight, such as 20 to 25 mg/kg bodyweight, for example 25 to 30 mg/kg bodyweight.

### Medical Use

It is an aspect of the present disclosure to provide a pharmaceutical composition, such as an oral formulation, comprising AP1189 free base, such as the crystalline form of AP1189 free base as disclosed herein for use in the treatment of a disease or disorder.

In some embodiments, the disease or disorder is selected from the group consisting of a kidney disease, an arthritic disease, a viral disease or disorder, and a cardiovascular disease and/or atherosclerosis.

### Kidney Disease

It is an aspect of the present disclosure to provide a pharmaceutical composition according to the present disclosure for use in treating or preventing a kidney disease.

Also disclosed is a method of treating or preventing a kidney disease in a subject in need thereof, wherein the subject is administered a therapeutically effect amount of the pharmaceutical composition of the present disclosure.

Also disclosed is the use of a pharmaceutical composition according to the present disclosure for use in the manufacture of a medicament for the treatment or prevention of a kidney disease.

In some embodiments said kidney disease present with proteinuria. In some embodiments said kidney disease is a proteinuric kidney disease.

In some embodiments said kidney disease is a glomerular disease

In some embodiments said kidney disease is nephrotic syndrome (glomerulonephrosis).

In some embodiments said kidney disease is primary nephrotic syndrome (primary glomerulonephrosis).

In some embodiments said primary nephrotic syndrome is membranous glomerulonephritis (MGN) (or membranous nephropathy (MN)).

In some embodiments said primary nephrotic syndrome is focal segmental glomerulosclerosis (FSGS).

In some embodiments said primary nephrotic syndrome is membranoproliferative glomerulonephritis (MPGN) (mesangiocapillary glomerulonephritis).

In some embodiments said membranoproliferative glomerulonephritis (MPGN) is selected from Type 1 MPGN and Type 2 MPGN.

In some embodiments said primary nephrotic syndrome is rapidly progressive glomerulonephritis (RPGN) (crescentic GN).

In some embodiments said primary nephrotic syndrome is minimal change disease (MCD).

In some embodiments said kidney disease is secondary nephrotic syndrome (secondary glomerulonephrosis).

In some embodiments said secondary nephrotic syndrome is caused by an underlying autoimmune disease, an underlying cancer disease, an underlying genetic disorder, or by an underlying disease selected from the group consisting of: Systemic lupus erythematosus (SLE), Diabetic nephropathy, Sarcoidosis, Sjögren's syndrome, Amyloidosis, Multiple myeloma, Vasculitis, Cancer and Genetic disorders (such as congenital nephrotic syndrome).

In some embodiments said secondary nephrotic syndrome is caused by Diabetic nephropathy, by an infection, such as a urinary tract infection, such as an infection selected from the group consisting of HIV, syphilis, hepatitis such as hepatitis A, B and C, post-streptococcal infection, urinary schistosomiasis and Ebola. In some embodiments said secondary nephrotic syndrome is drug-induced.

In some embodiments said kidney disease is an inflammatory kidney disease.

In some embodiments said kidney disease is glomerulonephritis (GN). In some embodiments said glomerulonephritis is selected from the group consisting of IgA nephropathy (Berger's disease), IgM nephropathy, Post-infectious glomerulonephritis and Thin basement membrane disease.

In some embodiment said kidney disease is idiopathic membranous nephropathy (iMN).

In some embodiments there is provided a pharmaceutical composition according to the present disclosure for use in treating or preventing idiopathic membranous nephropathy (iMN).

### Arthritic Disease

It is an aspect of the present disclosure to provide a pharmaceutical composition according to the present disclosure for use in treating or preventing an arthritic disease.

Also disclosed is a method of treating or preventing an arthritic disease in a subject in need thereof, wherein the subject is administered a therapeutically effect amount of the pharmaceutical composition of the present disclosure.

Also disclosed is the use of a pharmaceutical composition according to the present disclosure for use in the manufacture of a medicament for the treatment or prevention of an arthritic disease.

In one embodiment the arthritic disease is an auto-immune disease and/or an inflammatory disease that presents with joint inflammation.

In one embodiment, the arthritic disease is selected from the group consisting of inflammatory arthritis, degenerative arthritis, metabolic arthritis, reactive arthritis and infectious arthritis.

In one embodiment, the arthritic disease is inflammatory arthritis.

In one embodiment, the inflammatory arthritis is selected from the group consisting of Rheumatoid Arthritis (RA), Psoriatic Arthritis, and Ankylosing Spondylitis.

In one embodiment, the inflammatory arthritis is Rheumatoid Arthritis (RA).

In one embodiment, the rheumatoid arthritis is severe active RA (CDAI>22). In one embodiment, the rheumatoid arthritis is RA with a CDAI>22.

In one embodiment, the rheumatoid arthritis is RA with a DAS28 score of above 5.1.

In one embodiment, the rheumatoid arthritis is juvenile rheumatoid arthritis (JRA).

In one embodiment, the degenerative arthritis is osteoarthritis.

In one embodiment, the metabolic arthritis is gouty arthritis.

In one embodiment, the reactive and/or infectious arthritis is arthritis associated with infection with one or more of Hepatitis C, Chlamydia, gonorrhoea, salmonella or shigella.

In one embodiment the arthritic disease is arthritis as part of a systemic inflammatory disease.

In one embodiment, the arthritis as part of a systemic inflammatory disease, such as an inflammatory disease selected from the group consisting of systemic lupus erythematosus, mixed connective tissue disease, Still's disease, and Polymyalgia Rheumatica.

In some embodiments there is provided a pharmaceutical composition according to the present disclosure for use in treating or preventing rheumatoid arthritis.

### Viral Disease or Disorder

It is an aspect of the present disclosure to provide a pharmaceutical composition according to the present disclosure for use in treating or preventing a viral disease or disorder.

Also disclosed is a method of treating or preventing a viral disease or disorder in a subject in need thereof, wherein the subject is administered a therapeutically effect amount of the pharmaceutical composition of the present disclosure.

Also disclosed is the use of a pharmaceutical composition according to the present disclosure for use in the manufacture of a medicament for the treatment or prevention of a viral disease or disorder.

In some embodiments said viral disease or disorder is a symptomatic viral disease or disorder.

In some embodiments said viral disease or disorder is a symptomatic viral disease or disorder with inflammation, such as hyperinflammation.

In some embodiments said viral disease or disorder is a symptomatic viral disease or disorder with inflammation, such as hyperinflammation, in one or more organs. Inflammation in one or more organs may also be referred to as local inflammation.

In some embodiments said one or more organs are selected from the group consisting of lungs, the respiratory tract, kidney, liver, pancreas, spleen, exocrine glands, endocrine glands, lymph nodes, brain, heart, muscles, bone marrow, skin, skeleton, bladder, reproduction organs including the phallopian tubes, eye, ear, vascular system, the gastroinstestinal tract including small intestines, colon, rectum, canalis analis and the prostate gland.

In some embodiments said viral disease or disorder is inflammatory viral diseases or disorders.

In some embodiments said viral disease or disorder is a viral respiratory infection, such as a viral lower respiratory infection.

In some embodiments said viral disease or disorder is viral respiratory diseases or disorders.

In some embodiments said viral disease or disorder is viral diseases or disorders of the lung.

In some embodiments said viral disease or disorder is viral diseases or disorders with inflammation in the respiratory system, such as in the lungs and/or respiratory tract.

In some embodiments said viral disease or disorder is viral diseases or disorders with one or more respiratory symptoms. In one embodiment said one or more respiratory symptoms are selected from the group consisting of cough, dry cough, dyspnea, impaired oxygenation, respiratory illness, respiratory dysfunction, respiratory failure, respiratory syndrome and acute respiratory disease (ARD).

In some embodiments said viral disease or disorder is severe disease. Severe disease present with dyspnoea, increased respiratory frequency, reduced blood oxygen saturation and/or lung infiltrates.

In some embodiments said viral disease or disorder is critical disease. Critical disease present with respiratory failure, septic shock, and/or multiple organ dysfunction (MOD) or multiple organ failure (MOF).

In some embodiments said viral disease or disorder is viral pneumonia.

In some embodiments said viral disease or disorder is viral bronchiolitis.

In some embodiments said viral disease or disorder is viral diseases or disorders with respiratory failure.

In some embodiments said viral disease or disorder is acute respiratory distress syndrome (ARDS).

In some embodiments said viral disease or disorder is viral acute respiratory distress syndrome (ARDS).

In some embodiments said viral disease or disorder is symptomatic COVID-19 with acute respiratory distress syndrome (ARDS).

In some embodiments there is provided an oral formulation, a pharmaceutical composition, or unit dosage form according to the present disclosure for use in treating or preventing ARDS, such as viral ARDS.

In some embodiments said viral disease or disorder is viral diseases and disorders with systemic inflammatory distress syndrome (SIDS) and/or sepsis.

In some embodiments said viral disease or disorder is viral diseases and disorders with pulmonary insufficiency.

In some embodiments said viral disease or disorder is viral diseases or disorders with cytokine release syndrome (CRS) and/or a cytokine storm (hypercytokinemia).

In some embodiments said viral disease or disorder is caused by a viral infection selected from the group consisting of Severe Acute Respiratory Syndrome CoronaVirus 2 (SARS-CoV-2), often referred to as the COVID-19 virus; SARS-CoV, MERS-CoV, the dengue virus and influenza virus (including Type A, Type B and Type C).

Cardiovascular Disease and/or Atherosclerosis

It is an aspect of the present disclosure to provide a pharmaceutical composition according to the present disclosure for use in treating or preventing a cardiovascular disease and/or atherosclerosis.

Also disclosed is a method of treating or preventing a cardiovascular disease and/or atherosclerosis in a subject in need thereof, wherein the subject is administered a therapeutically effect amount of the pharmaceutical composition of the present disclosure.

Also disclosed is the use of a pharmaceutical composition according to the present disclosure for use in the manufacture of a medicament for the treatment or prevention of a cardiovascular disease and/or atherosclerosis.

In some embodiments said cardiovascular disease is selected from the group consisting of coronary artery diseases (CAD) such as angina and myocardial infarction (commonly known as a heart attack); stroke, heart failure, hypertensive heart disease, rheumatic heart disease, cardiomyopathy, abnormal heart rhythms, congenital heart disease, valvular heart disease, carditis, aortic aneurysms, peripheral artery disease, vascular disease, thromboembolic disease, and venous thrombosis.

In some embodiments said cardiovascular disease is atherosclerotic cardiovascular disease.

In some embodiments said atherosclerotic cardiovascular disease is selected from the group consisting of coronary artery disease, stroke (cerebrovascular disease), and peripheral artery disease.

In some embodiments said cardiovascular disease is vascular inflammation.

### Systemic Inflammatory Disorders

It is an aspect of the present disclosure to provide a pharmaceutical composition according to the present disclosure for use in treating or preventing a systemic inflammatory disorder.

Also disclosed is a method of treating or preventing a systemic inflammatory disorder in a subject in need thereof, wherein the subject is administered a therapeutically effect amount of the pharmaceutical composition of the present disclosure.

Also disclosed is the use of a pharmaceutical composition according to the present disclosure for use in the manufacture of a medicament for the treatment or prevention of a systemic inflammatory disorder.

Systemic disorders with possible involvement of the nervous system include a variety of diseases with presumed inflammatory and autoimmune pathomechanisms, among them Behçet disease, sarcoidosis, systemic lupus erythematosus, juvenile idiopathic arthritis, scleroderma, and Sjögren syndrome. This disease group encompasses systemic inflammatory disorders with a genetically defined dysregulation of the innate immune system as well as systemic autoimmune disorders characterized by alterations of the adaptive immunity such as autoantibodies and autoreactive T cells.

In some embodiments said systemic inflammatory disorder is an autoimmune disorder.

In some embodiments said systemic inflammatory disorder is selected from the group consisting of Behçet disease, sarcoidosis, systemic lupus erythematosus, juvenile idiopathic arthritis, scleroderma, Sjögren syndrome, myositis including dermamyositis and polymyositis, vasculitis, giant cell arteritis, ankylosing spondylitis, polymyalgia rheumatic and psoriatic arthritis.

### Examples

### Example 1: API189 Freebase Preparations

### Method 1

A salt break for preparation of the free base from the acetate starting material was carried out as follows using extraction into ethyl acetate: 35 mL of ethyl acetate and 35 mL of 1 M sodium bicarbonate was added to ca. 500 mg of AP1189 acetate. The mixture was transferred to a separating funnel and the aqueous layer was removed and 15 mL of water was added to wash the organic layer. The phases were separated and, and sodium sulfate was added to the organic phase and left for ca. 10 minutes then the slurry was filtered by Büchner filtration using grade 1 filter paper. The solution was transferred to a round bottom flask and the solvent removed via rotary evaporation. 15 mL of DCM was added to the prepared freebase to dissolve, and the solution was left to evaporate at ambient temperature and pressure for ca. 18 h. The solid was dried under vacuum for ca. 1 h. The solids were analysed by XRPD, HPLC for purity and KF to determine the water content.

### Results of Method 1

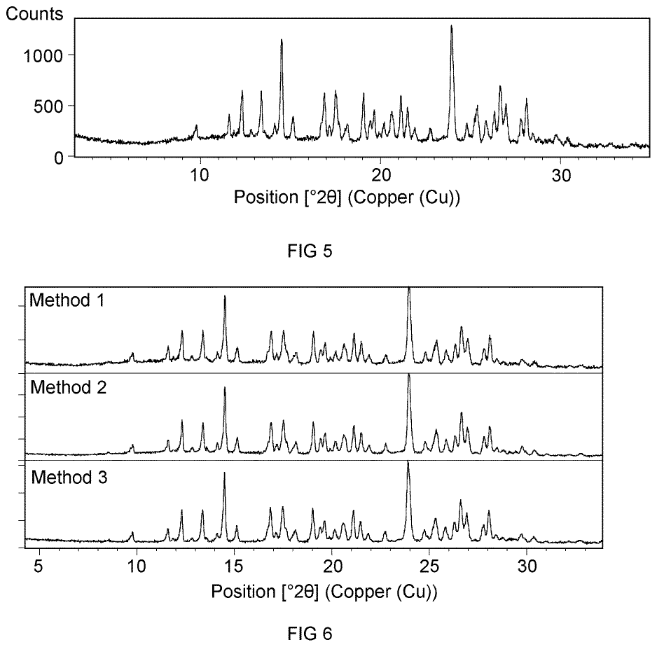

FIGS. 5 and 6 show the XRPD analysis of the material obtained. This was designated as freebase Pattern 1. KF analysis measured 0.3% w/w water for freebase Pattern 1.

### Method 2

A 2 g salt break was carried out as follows: 140 mL of ethyl acetate and 140 mL of 1 M sodium bicarbonate was added to ca. 2 g of AP1189 acetate. The mixture was transferred to a separating funnel and the organic layer was washed with 50 ml water. The organic phase was druid with sodium sulfate for ca. 10 minutes, and the solids were removed using grade 1 filter paper. The solvent was removed by rotary evaporation. The residue was dissolved in 60 mL of DCM, and the solvent was left to evaporate at ambient temperature and pressure for ca. 17 h. The solids were then dried under vacuum for ca. 1 h at ambient temperature. The solids were then analysed by XRPD.

### Results of Method 2

FIG. 6 shows the XRPD analysis of the material obtained. This corresponded to freebase Pattern 1.

### Method 3

AP1189 freebase was prepared using a similar method as Method 2 above, starting from 2.8 g AP1189 acetate, using 196 ml of ethyl acetate and 196 ml of 1 M sodium bicarbonate, washing with 84 ml water, and recrystallising from 70 ml of DMC.

### Results of Method 3

FIG. 6 shows the XRPD analysis of the material obtained. This corresponded to freebase Pattern 1.

### Example 2: Polymorphism Assessment for AP1189 Free Base

### Methods

An initial polymorphism assessment was carried out in order to identify any potential alternate forms. The initial polymorphism assessment was carried out as follows: To ca. 7 mg of freebase Pattern 1 from DCM evaporation the appropriate solvent system was added in aliquots of 50 μL and if solid remained the vial was gently heated to ca. 40 °C to aid dissolution. Solvent addition was continued until the material had fully dissolved or 1 mL of the appropriate solvent system had been added (<10 mg/mL). Anti-solvent (t-BME) was added dropwise (with stirring) at ambient. The volumes of t-BME used are shown in Table 1. If the solutions were already present as slurries prior to addition of anti-solvent, no anti-solvent was added. The experiments were temperature cycled between ambient temperature and 40 °C in 4 hour cycles for ca. 24 h with agitation. Slurries were isolated by centrifugation and solutions allowed to evaporate at ambient temperature. XRPD analysis was carried out for the solids which were obtained.

### Results

Table 1 shows the volumes of anti-solvent used in additions.

TABLE 1
Anti-solvent volumes used for anti-solvent additions where
anti-solvent necessary.

| | | Volume of |
|---|---|---|
| | | Anti-Solvent |
| Number | Solvent System | Added (μL) |
| 1 | 2-Methyl THF | 650 |
| 3 | 90% 2-Propanol: | 650 |
| | 10% Water (% v/v) | |
| | (calculated a_{w} 0.6) | |
| 4 | 98.5% Ethanol: 1.5% | 450 |
| | Water (% v/v) | |
| | (calculated a_{w} 0.2) | |
| 5 | Acetone | 150 |
| 6 | Acetonitrile | 250 |
| 8 | Dimethylsulfoxide | 50 |
| 9 | Ethanol | 300 |
| 12 | Methanol | 300 |
| 16 | Tetrahydrofuran | 250 |

Table 2 shows the results of the polymorphism assessment.

TABLE 2
Initial polymorphism assessment results of AP1189 freebase

| | | | | XRPD |
|---|---|---|---|---|
| | | | | Analysis |
| | | | | after |
| | | | | Temper- |
| Num- | | Anti- | Solubility | ature |
| ber | Solvent System | Solvent | (mg/mL) | Cycling |
| 1 | 2-Methyl THF | t-BME | ≥10 | Pattern 1 |
| 2 | 2-Propanol | N/A | <7 | Glass |
| 3 | 90% 2-Propanol: | t-BME | ≥10 | Glass |
| | 10% Water (% | | | |
| | v/v) (calculated | | | |
| | a_{w} 0.6) | | | |
| 4 | 98.5% Ethanol : | t-BME | ≥15 | Glass |
| | 1.5% Water (% | | | |
| | v/v) (calculated | | | |
| | a_{w} 0.2) | | | |
| 5 | Acetone | t-BME | ≥49 | Pattern 1 |
| 6 | Acetonitrile | t-BME | ≥27 | Pattern 1 |
| 7 | Dichloromethane | N/A | <7 | Pattern 1 |
| 8 | Dimethylsulfoxide | t-BME | ≥136 | Solution |
| 9 | Ethanol | t-BME | ≥13 | Glass |
| 10 | Ethyl Acetate | N/A | <7 | Pattern 1 |
| 11 | Heptane | N/A | <7 | Pattern 1 |
| 12 | Methanol | t-BME | ≥142 | Pattern 1 |
| 13 | Methylethyl Ketone | N/A | ≥17 | Glass |
| 14 | Methylisobutyl Ketone | N/A | <7 | Pattern 1 |
| 15 | tert-Butylmethyl ether | N/A | <7 | Pattern 1 |
| 16 | Tetrahydrofuran | t-BME | ≥35 | Glass |
| 17 | Toluene | N/A | <7 | Pattern 1 |
| 18 | Water | N/A | <7 | Pattern 1 |

The AP1189 freebase Pattern 3 DMSO solvate was prepared as follows: Dimethylsulfoxide (25 μL) was added to ca. 100 mg of amorphous free base in a 20 mL scintillation vial equipped with a magnetic stirring bar. The resulting slurry was thermally cycled for 3 days between 40 °C and 20 °C (Ramp rate: 0.1 °C/min with isothermal holds of 1 hour at 40 °C and 20 °C). Solids were analysed by XRPD.

The AP1189 freebase Pattern 4 DMF solvate was prepared as follows: Dimethylformamide (25 μL) was added to ca. 100 mg of amorphous free base in a 20 mL scintillation vial equipped with a magnetic stirring bar. The resulting slurry was thermally cycled for 3 days between 40 °C and 20 °C (Ramp rate: 0.1 °C/min with isothermal holds of 1 hour at 40 °C and 20 °C). Solids were analysed by XRPD.

The AP1189 freebase Pattern 5 NMP solvate was prepared as follows: N-Methylpyrrolidone (25 μL) was added to ca. 100 mg of amorphous free base in a 20 mL scintillation vial equipped with a magnetic stirring bar. The resulting slurry was thermally cycled for 3 days between 40 °C and 20 °C (Ramp rate: 0.1 °C/min with isothermal holds of 1 hour at 40 °C and 20 °C). Solids were analysed by XRPD.

### Example 3: X-ray Powder Diffraction

### Methods

XRPD analysis was carried out on a PANalytical X'pert pro with PIXcel detector (128 channels), scanning the samples between 3 and 35° 2θ. The material was gently ground (where required) to release any agglomerates and loaded onto a multi-well plate with Kapton or Mylar polymer film to support the sample. The multi-well plate was then placed into the diffractometer and analysed using Cu K radiation (α1λ=1.54060 Å; α2=1.54443 Å; β=1.39225 Å; α1:α2 ratio=0.5) running in transmission mode (step size 0.0130° 20, step time 18.87 s) using 40 kV/40 mA generator settings.

### Results

The XRPD diffractogram for AP1189 freebase Pattern 1 crystallised from DCM is shown in FIG. 5. The corresponding XRPD diffractogram peak list for freebase Pattern 1 is shown in Table 3a.

TABLE 3a
XRPD diffractogram peak list for freebase Pattern 1 from DCM.
Characteristic peaks are indicated in bold.

| Pos. | Height | FWHM | d-spacing | Rel. Int. |
|---|---|---|---|---|
| [°2θ] | [cts] | Left [°2θ] | [Å] | [%] |
| 8.5374 | 65.40 | 0.1535 | 10.35729 | 4.70 |
| 9.7987 | 249.10 | 0.0640 | 9.02676 | 17.89 |
| 11.6310 | 324.43 | 0.0640 | 7.60851 | 23.31 |
| 11.8713 | 137.06 | 0.0768 | 7.45506 | 9.85 |
| 12.3242 | 681.78 | 0.0768 | 7.18209 | 48.98 |
| 12.8610 | 159.28 | 0.1023 | 6.88347 | 11.44 |
| 13.4113 | 688.28 | 0.0895 | 6.60225 | 49.44 |
| 13.5979 | 92.73 | 0.0900 | 6.50668 | 6.66 |
| 14.1466 | 271.07 | 0.0384 | 6.26070 | 19.47 |
| 14.5427 | 1392.03 | 0.0895 | 6.09107 | 100.00 |
| 15.1438 | 410.91 | 0.0895 | 5.85063 | 29.52 |
| 16.9080 | 711.29 | 0.0895 | 5.24391 | 51.10 |
| 17.2102 | 235.93 | 0.0895 | 5.15252 | 16.95 |
| 17.5503 | 743.52 | 0.1023 | 5.05343 | 53.41 |
| 18.1743 | 306.90 | 0.0512 | 4.88130 | 22.05 |
| 19.0649 | 748.48 | 0.0768 | 4.65525 | 53.77 |
| 19.4208 | 318.33 | 0.0512 | 4.57072 | 22.87 |
| 19.6847 | 476.44 | 0.1023 | 4.51005 | 34.23 |
| 20.1632 | 324.29 | 0.0512 | 4.40409 | 23.30 |
| 20.6141 | 424.48 | 0.2047 | 4.30875 | 30.49 |
| 21.1809 | 635.23 | 0.1151 | 4.19471 | 45.63 |
| 21.5468 | 449.04 | 0.1151 | 4.12431 | 32.26 |
| 21.9549 | 218.66 | 0.0512 | 4.04856 | 15.71 |
| 22.7821 | 245.72 | 0.1279 | 3.90339 | 17.65 |
| 23.9014 | 1120.09 | 0.1407 | 3.72307 | 80.46 |
| 24.7877 | 270.26 | 0.0640 | 3.59192 | 19.42 |
| 25.4291 | 480.46 | 0.1407 | 3.50276 | 34.51 |
| 25.8885 | 354.63 | 0.0768 | 3.44164 | 25.48 |
| 26.3129 | 400.38 | 0.0512 | 3.38708 | 28.76 |
| 26.6522 | 793.60 | 0.1535 | 3.34473 | 57.01 |
| 27.0189 | 613.85 | 0.1279 | 3.30016 | 44.10 |
| 27.4120 | 45.81 | 0.0900 | 3.25103 | 3.29 |
| 27.8366 | 403.36 | 0.0640 | 3.20505 | 28.98 |
| 28.0782 | 573.39 | 0.1151 | 3.17802 | 41.19 |
| 28.4957 | 138.79 | 0.1279 | 3.13240 | 9.97 |
| 28.8482 | 105.15 | 0.1535 | 3.09492 | 7.55 |
| 29.1649 | 85.95 | 0.1023 | 3.06204 | 6.17 |
| 29.4304 | 74.82 | 0.0900 | 3.03250 | 5.37 |
| 29.7663 | 194.58 | 0.0768 | 3.00152 | 13.98 |
| 30.4142 | 100.77 | 0.1791 | 2.93904 | 7.24 |
| 31.1060 | 35.01 | 0.2047 | 2.87524 | 2.51 |
| 32.2720 | 35.30 | 0.1535 | 2.77397 | 2.54 |
| 32.7706 | 49.77 | 0.2047 | 2.73289 | 3.58 |
| 34.0785 | 30.78 | 0.3070 | 2.63094 | 2.21 |
| 34.7416 | 38.05 | 0.0768 | 2.58223 | 2.73 |

The XRPD diffractogram for AP1189 freebase Pattern 3 DMSO solvate is shown in FIG. 7. The corresponding XRPD diffractogram peak list for freebase Pattern 3 is shown in Table 3b.

TABLE 3b
XRPD diffractogram peak list for freebase Pattern 3 DMSO solvate.
Characteristic peaks are indicated in bold.

| Pos. | Height | d-spacing | Rel. Int. |
|---|---|---|---|
| [°2θ] | [cts] | [Å] | [%] |
| 4.5306 | 126.55 | 19.50408 | 18.38 |
| 12.2988 | 24.88 | 7.19091 | 3.61 |
| 13.1891 | 78.37 | 6.70742 | 11.38 |
| 13.3667 | 110.71 | 6.61871 | 16.08 |
| 13.6225 | 179.84 | 6.50035 | 26.12 |
| 14.3484 | 284.94 | 6.1731 | 41.38 |
| 15.0165 | 254.65 | 5.89993 | 36.98 |
| 15.513 | 312.33 | 5.71219 | 45.36 |
| 16.2564 | 59.54 | 5.44812 | 8.65 |
| 16.6854 | 688.15 | 5.31337 | 99.94 |
| 16.9828 | 94.22 | 5.21667 | 13.68 |
| 17.6691 | 249.13 | 5.01971 | 36.18 |
| 18.3894 | 317.58 | 4.8247 | 46.12 |
| 18.807 | 47.7 | 4.71459 | 6.93 |
| 19.6479 | 688.57 | 4.51841 | 100 |
| 20.2663 | 160.71 | 4.3819 | 23.34 |
| 20.7462 | 239.43 | 4.28163 | 34.77 |
| 21.0386 | 504.27 | 4.22277 | 73.23 |
| 21.4061 | 36.36 | 4.14766 | 5.28 |
| 21.989 | 201.43 | 4.04236 | 29.25 |
| 22.4452 | 145.94 | 3.96121 | 21.19 |
| 22.8277 | 222.72 | 3.8957 | 32.34 |
| 23.5476 | 421.65 | 3.7782 | 61.23 |
| 23.9211 | 263.52 | 3.72005 | 38.27 |
| 24.216 | 157.82 | 3.67542 | 22.92 |
| 24.8072 | 37.96 | 3.58618 | 5.51 |
| 25.7767 | 51.74 | 3.45631 | 7.51 |
| 26.3408 | 18.99 | 3.38076 | 2.76 |
| 26.5668 | 54.62 | 3.35251 | 7.93 |
| 26.9704 | 24.23 | 3.30325 | 3.52 |
| 28.9088 | 48.83 | 3.08857 | 7.09 |
| 29.7264 | 64.49 | 3.00546 | 9.37 |
| 30.1207 | 31.35 | 2.96701 | 4.55 |

The XRPD diffractogram for AP1189 freebase Pattern 4 DMF solvate is shown in FIG. 8. The corresponding XRPD diffractogram peak list for freebase Pattern 4 is shown in Table 3c.

TABLE 3c
XRPD diffractogram peak list for freebase Pattern 4 DMF solvate.
Characteristic peaks are indicated in bold.

| Pos. | Height | d-spacing | Rel. Int. |
|---|---|---|---|
| [°2θ] | [cts] | [Å] | [%] |
| 4.5986 | 31.21 | 19.20009 | 3.82 |
| 11.4125 | 39.89 | 7.74727 | 4.88 |
| 12.2764 | 176.94 | 7.20991 | 21.67 |
| 12.8558 | 198.64 | 6.88625 | 24.32 |
| 13.3842 | 158.41 | 6.61555 | 19.4 |
| 13.9631 | 78.8 | 6.33733 | 9.65 |
| 14.5674 | 279.94 | 6.0808 | 34.28 |
| 15.101 | 80.02 | 5.86224 | 9.8 |
| 15.4147 | 217.85 | 5.7484 | 26.67 |
| 16.2246 | 554.62 | 5.46324 | 67.91 |
| 16.8687 | 191.97 | 5.25604 | 23.51 |
| 17.5111 | 210.98 | 5.06465 | 25.83 |
| 17.8156 | 107.23 | 4.97464 | 13.13 |
| 17.9405 | 236.66 | 4.94438 | 28.98 |
| 19.0495 | 148.8 | 4.65511 | 18.22 |
| 19.5176 | 775.43 | 4.54828 | 94.95 |
| 19.831 | 167.47 | 4.47339 | 20.51 |
| 20.3934 | 264.92 | 4.35489 | 32.44 |
| 20.9952 | 491.33 | 4.2314 | 60.16 |
| 21.609 | 306.31 | 4.11257 | 37.51 |
| 22.4908 | 234.36 | 3.95002 | 28.7 |
| 22.7101 | 263.84 | 3.91236 | 32.31 |
| 22.9112 | 462.5 | 3.88169 | 56.63 |
| 23.1947 | 816.68 | 3.83488 | 100 |
| 23.3559 | 672.37 | 3.80879 | 82.33 |
| 23.9909 | 361.75 | 3.70939 | 44.3 |
| 25.0696 | 776.33 | 3.55218 | 95.06 |
| 25.3271 | 200.2 | 3.51373 | 24.51 |
| 25.6561 | 146.23 | 3.46941 | 17.91 |
| 25.9747 | 195.8 | 3.43041 | 23.97 |
| 26.6627 | 226.33 | 3.34344 | 27.71 |
| 26.9449 | 145.98 | 3.30632 | 17.88 |
| 28.1133 | 172.42 | 3.17413 | 21.11 |
| 28.6449 | 98.07 | 3.11384 | 12.01 |
| 28.7958 | 120.51 | 3.09787 | 14.76 |
| 29.2033 | 159.39 | 3.0581 | 19.52 |
| 30.076 | 139.86 | 2.97131 | 17.12 |
| 30.6878 | 37.82 | 2.91106 | 4.63 |
| 31.6947 | 354.14 | 2.82317 | 43.36 |
| 32.2661 | 184.9 | 2.77446 | 22.64 |
| 33.6563 | 136.83 | 2.66297 | 16.75 |
| 34.3214 | 146.38 | 2.61288 | 17.92 |

The XRPD diffractogram for AP1189 freebase Pattern 5 NMP solvate is shown in FIG. 9. The corresponding XRPD diffractogram peak list for freebase Pattern 5 is shown in Table 3d.

TABLE 3d
XRPD diffractogram peak list for freebase Pattern 5 NMP solvate.
Characteristic peaks are indicated in bold.

| Pos. | Height | d-spacing | Rel. Int. |
|---|---|---|---|
| [°2θ] | [cts] | [Å] | [%] |
| 11.7253 | 91.88 | 7.54128 | 4.99 |
| 12.0512 | 141.47 | 7.33809 | 7.68 |
| 12.2991 | 812.21 | 7.1967 | 44.08 |
| 12.5588 | 199.48 | 7.04846 | 10.83 |
| 12.7543 | 137.07 | 6.93507 | 7.44 |
| 13.0042 | 286.03 | 6.80801 | 15.52 |
| 13.9816 | 295.18 | 6.33422 | 16.02 |
| 14.4777 | 748.15 | 6.11824 | 40.6 |
| 14.9496 | 90.89 | 5.92128 | 4.93 |
| 15.5122 | 949.91 | 5.71249 | 51.55 |
| 16.157 | 1702.03 | 5.48593 | 92.37 |
| 16.3876 | 171.2 | 5.40478 | 9.29 |
| 16.7477 | 206.11 | 5.28936 | 11.19 |
| 16.9371 | 356.66 | 5.23497 | 19.36 |
| 17.1934 | 675.17 | 5.15751 | 36.64 |
| 17.3304 | 690.88 | 5.11705 | 37.49 |
| 17.8905 | 514.46 | 4.95809 | 27.92 |
| 18.5217 | 391.91 | 4.79053 | 21.27 |
| 19.0004 | 422.29 | 4.6709 | 22.92 |
| 19.3384 | 1157.22 | 4.59002 | 62.8 |
| 19.6182 | 277.62 | 4.52517 | 15.07 |
| 20.1299 | 768.73 | 4.4113 | 41.72 |
| 20.3728 | 502.62 | 4.35924 | 27.28 |
| 20.8207 | 792.62 | 4.26646 | 43.02 |
| 21.1742 | 325.69 | 4.19602 | 17.68 |
| 21.5926 | 597.85 | 4.11567 | 32.45 |
| 22.2748 | 416.93 | 3.99114 | 22.63 |
| 22.5459 | 555.85 | 3.94376 | 30.17 |
| 22.8011 | 1501.32 | 3.90019 | 81.48 |
| 23.22 | 1842.63 | 3.83076 | 100 |
| 23.6285 | 734.64 | 3.76545 | 39.87 |
| 24.481 | 221.96 | 3.63322 | 12.05 |
| 24.7722 | 469.08 | 3.59414 | 25.46 |
| 25.0847 | 423.86 | 3.55007 | 23 |
| 25.7325 | 318.72 | 3.46215 | 17.3 |
| 26.0931 | 142.76 | 3.41229 | 7.75 |
| 26.5905 | 101.83 | 3.34958 | 5.53 |
| 27.105 | 92.29 | 3.28716 | 5.01 |
| 27.8424 | 87.63 | 3.20174 | 4.76 |
| 28.3226 | 117.09 | 3.14854 | 6.35 |
| 28.5667 | 296.19 | 3.12477 | 16.07 |
| 28.9837 | 257.56 | 3.08076 | 13.98 |
| 29.5539 | 241.79 | 3.02261 | 13.12 |
| 29.9145 | 315.62 | 2.98699 | 17.13 |
| 30.6129 | 116.5 | 2.92041 | 6.32 |
| 32.0618 | 101.28 | 2.79168 | 5.5 |
| 33.161 | 29.58 | 2.70161 | 1.61 |
| 34.0534 | 36.28 | 2.63283 | 1.97 |

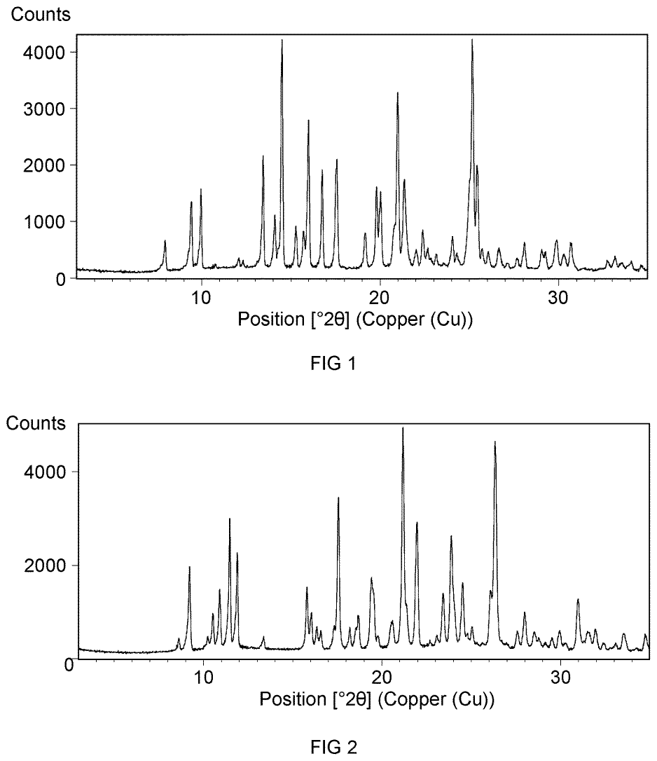

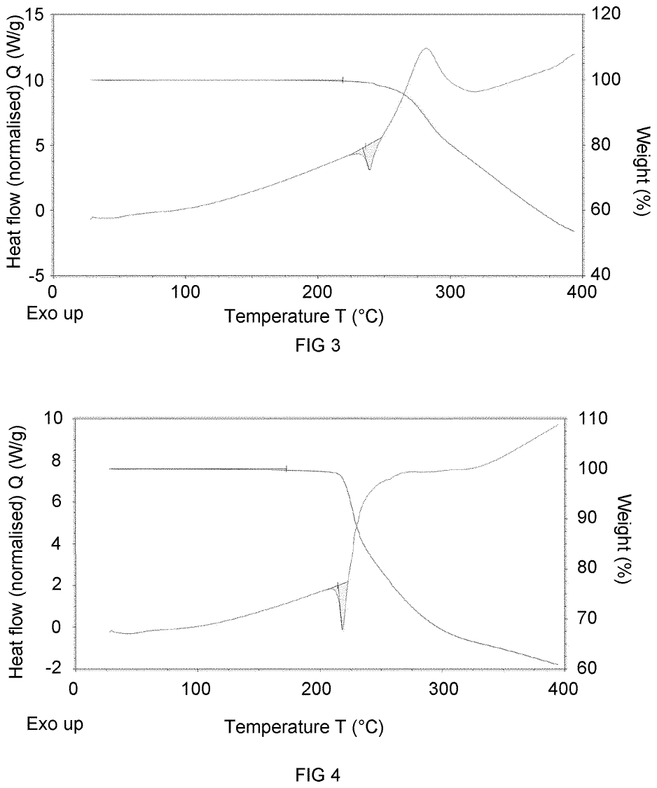

The XRPD diffractogram for AP1189 tosylate salt Pattern 1 crystallised from methanol is shown in FIG. 1. The corresponding XRPD diffractogram peak list for tosylate salt

TABLE 4
XRPD diffractogram peak list for tosylate Pattern 1 from methanol.
Characteristic peaks are indicated in bold.

| Pos. | Height | FWHM Left | d-spacing | Rel. Int. |
|---|---|---|---|---|
| [°2θ] | [cts] | [°2θ] | [Å] | [%] |
| 7.95280 | 565.46 | 0.0768 | 11.11733 | 13.44 |
| 9.42330 | 1279.54 | 0.0895 | 9.38555 | 30.41 |
| 9.96040 | 1509.46 | 0.0895 | 8.88062 | 35.88 |
| 10.76630 | 133.52 | 0.0768 | 8.21756 | 3.17 |
| 12.07190 | 246.37 | 0.0640 | 7.33164 | 5.86 |
| 12.31920 | 203.44 | 0.0640 | 7.18500 | 4.84 |
| 13.44230 | 2110.32 | 0.0895 | 6.58708 | 50.16 |
| 14.09060 | 1043.18 | 0.0768 | 6.28546 | 24.80 |
| 14.49790 | 4207.16 | 0.1023 | 6.10978 | 100.00 |
| 15.28210 | 833.29 | 0.1023 | 5.79797 | 19.81 |
| 15.70980 | 713.17 | 0.0895 | 5.64106 | 16.95 |
| 15.98490 | 2747.52 | 0.1023 | 5.54462 | 65.31 |
| 16.74560 | 1838.99 | 0.1023 | 5.29441 | 43.71 |
| 17.55870 | 1963.25 | 0.1535 | 5.05103 | 46.66 |
| 19.15130 | 708.39 | 0.1151 | 4.63442 | 16.84 |
| 19.79500 | 1554.81 | 0.1023 | 4.48515 | 36.96 |
| 20.01010 | 1455.65 | 0.1151 | 4.43743 | 34.60 |
| 20.74200 | 698.27 | 0.1023 | 4.28247 | 16.60 |
| 20.98000 | 3263.54 | 0.1279 | 4.23443 | 77.57 |
| 21.34790 | 1658.88 | 0.1279 | 4.16229 | 39.43 |
| 22.02470 | 407.91 | 0.1151 | 4.03589 | 9.70 |
| 22.38540 | 763.27 | 0.0640 | 3.97167 | 18.14 |
| 22.65840 | 400.17 | 0.0384 | 3.92442 | 9.51 |
| 22.83650 | 258.14 | 0.0768 | 3.89421 | 6.14 |
| 23.14460 | 310.98 | 0.1279 | 3.84307 | 7.39 |
| 23.55690 | 160.13 | 0.1279 | 3.77674 | 3.81 |
| 24.05140 | 646.06 | 0.1023 | 3.70020 | 15.36 |
| 24.29100 | 327.31 | 0.1023 | 3.66424 | 7.78 |
| 25.15830 | 4097.87 | 0.1151 | 3.53984 | 97.40 |
| 25.43100 | 1927.26 | 0.1151 | 3.50250 | 45.81 |
| 25.70240 | 416.16 | 0.1023 | 3.46613 | 9.89 |
| 26.05180 | 374.86 | 0.1023 | 3.42043 | 8.91 |
| 26.65210 | 421.78 | 0.1791 | 3.34474 | 10.03 |
| 27.13070 | 158.43 | 0.1535 | 3.28682 | 3.77 |
| 27.66440 | 258.67 | 0.0895 | 3.22461 | 6.15 |
| 28.07950 | 526.46 | 0.0895 | 3.17788 | 12.51 |
| 29.04430 | 391.90 | 0.1023 | 3.07448 | 9.32 |
| 29.24250 | 336.72 | 0.0384 | 3.05409 | 8.00 |
| 29.88270 | 587.69 | 0.1023 | 2.99009 | 13.97 |
| 30.28370 | 319.89 | 0.2303 | 2.95141 | 7.60 |
| 30.67120 | 509.33 | 0.1535 | 2.91500 | 12.11 |
| 31.39270 | 66.82 | 0.2047 | 2.84963 | 1.59 |
| 32.74010 | 194.37 | 0.1279 | 2.73537 | 4.62 |
| 33.15360 | 274.41 | 0.1791 | 2.70220 | 6.52 |
| 33.52400 | 157.80 | 0.1791 | 2.67318 | 3.75 |
| 34.06450 | 185.09 | 0.1279 | 2.63200 | 4.40 |
| 34.63470 | 112.70 | 0.1535 | 2.58996 | 2.68 |

The XRPD diffractogram for AP1189 fumarate salt Pattern 1 crystallised from isopropylalcohol:water 90:10 v/v is shown in FIG. 2. The corresponding XRPD diffractogram peak list for fumarate salt Pattern 1 from isopropylalcohol:water 90:10 v/v is shown in Table 5.

TABLE 5
XRPD diffractogram peak list for fumarate Pattern 1 from
isopropylalcohol:water 90:10 v/v. Characteristic peaks are
indicated in bold.

| Pos. | Height | FWHM Left | d-spacing | Rel. Int. |
|---|---|---|---|---|
| [°2θ] | [cts] | [°2θ] | [Å] | [%] |
| 8.6209 | 310.50 | 0.0640 | 10.25719 | 6.33 |
| 9.2296 | 1873.51 | 0.0895 | 9.58209 | 38.22 |
| 10.2345 | 347.27 | 0.0640 | 8.64332 | 7.08 |
| 10.5354 | 852.43 | 0.0640 | 8.39713 | 17.39 |
| 10.9191 | 1388.14 | 0.0768 | 8.10292 | 28.32 |
| 11.4728 | 2930.97 | 0.0895 | 7.71311 | 59.80 |
| 11.8926 | 2170.42 | 0.0895 | 7.44176 | 44.28 |
| 13.3718 | 346.23 | 0.0512 | 6.62169 | 7.06 |
| 15.7963 | 1426.98 | 0.1023 | 5.61039 | 29.11 |
| 16.0311 | 849.53 | 0.0895 | 5.52872 | 17.33 |
| 16.3575 | 561.06 | 0.1023 | 5.41913 | 11.45 |
| 16.5708 | 474.42 | 0.0768 | 5.34985 | 9.68 |
| 17.2941 | 531.10 | 0.0640 | 5.12771 | 10.84 |
| 17.5600 | 3319.60 | 0.1023 | 5.05067 | 67.73 |
| 18.1912 | 548.58 | 0.0895 | 4.87682 | 11.19 |
| 18.5329 | 529.75 | 0.0512 | 4.78765 | 10.81 |
| 18.6734 | 794.04 | 0.0895 | 4.75195 | 16.20 |
| 19.4122 | 1637.66 | 0.1023 | 4.57272 | 33.41 |
| 19.5565 | 1187.94 | 0.0512 | 4.53932 | 24.24 |
| 19.7714 | 359.40 | 0.0640 | 4.49046 | 7.33 |
| 20.5917 | 692.05 | 0.0512 | 4.31338 | 14.12 |
| 21.1710 | 4901.56 | 0.1279 | 4.19667 | 100.00 |
| 21.3706 | 1056.31 | 0.0768 | 4.15791 | 21.55 |
| 21.9494 | 2839.37 | 0.1407 | 4.04957 | 57.93 |
| 22.6944 | 230.40 | 0.1023 | 3.91828 | 4.70 |
| 23.0753 | 363.82 | 0.0895 | 3.85446 | 7.42 |
| 23.4284 | 1295.19 | 0.1407 | 3.79716 | 26.42 |
| 23.8881 | 2543.55 | 0.1535 | 3.72511 | 51.89 |
| 24.5122 | 1525.50 | 0.1535 | 3.63167 | 31.12 |
| 24.7719 | 401.96 | 0.0768 | 3.59418 | 8.20 |
| 25.0387 | 542.09 | 0.1151 | 3.55649 | 11.06 |
| 26.0822 | 1360.04 | 0.1279 | 3.41652 | 27.75 |
| 26.3417 | 4606.74 | 0.1407 | 3.38345 | 93.99 |
| 26.9772 | 178.54 | 0.0900 | 3.30243 | 3.64 |
| 27.5829 | 462.97 | 0.1023 | 3.23395 | 9.45 |
| 27.9868 | 874.09 | 0.1023 | 3.18819 | 17.83 |
| 28.5342 | 442.65 | 0.1151 | 3.12826 | 9.03 |
| 28.7849 | 321.79 | 0.1023 | 3.10159 | 6.57 |
| 29.1336 | 199.54 | 0.1279 | 3.06525 | 4.07 |
| 29.5204 | 318.82 | 0.0640 | 3.02597 | 6.50 |
| 29.9470 | 467.63 | 0.0895 | 2.98382 | 9.54 |
| 30.2812 | 204.98 | 0.1535 | 2.95165 | 4.18 |
| 30.9714 | 1155.91 | 0.1404 | 2.88504 | 23.58 |
| 31.0214 | 1083.09 | 0.0624 | 2.88766 | 22.10 |
| 31.5455 | 443.03 | 0.2808 | 2.83383 | 9.04 |
| 31.9500 | 514.93 | 0.0780 | 2.79887 | 10.51 |
| 32.4106 | 193.90 | 0.1872 | 2.76014 | 3.96 |
| 33.0815 | 204.69 | 0.1248 | 2.70568 | 4.18 |
| 33.5275 | 415.89 | 0.0936 | 2.67070 | 8.48 |
| 34.2307 | 89.28 | 0.1872 | 2.61743 | 1.82 |
| 34.7358 | 407.34 | 0.0468 | 2.58051 | 8.31 |

An AP1189 freebase sample lyophilized from a frozen 1,4-dioxane solution was amorphous as evidenced by an XRPD spectrum absent of any characteristic peaks.

### Conclusion

X-ray powder diffraction data was collected for a selection of different AP1189 salts and AP1189 freebase.

### Example 4: Thermogravimetric Analysis/Differential Scanning Calorimetry and Differential Scanning Calorimetry

### Methods

For the TGA/DSC assessment, approximately, 5-10 mg of material was added into a pre-tared open aluminium pan, loaded into a TA Instruments Discovery SDT 650 Auto-Simultaneous DSC and held at room temperature. The sample was then heated at a rate of 10 °C/min from 30 °C to 400 °C during which time the change in sample weight was recorded along with the heat flow response (DSC). Nitrogen was used as the sample purge gas, at a flow rate of 200 cm³/min.

For the DSC assessment, approximately, 1-5 mg of material was weighed into an aluminium DSC pan and sealed non-hermetically with an aluminium lid. The sample pan was then loaded into a TA Instruments Discovery DSC 2500 differential scanning calorimeter equipped with a RC90 cooler. The sample and reference were heated to 230 °C or 240 °C at a scan rate of 10 °C/min and the resulting heat flow response monitored. The sample was re-cooled to 20 °C and then reheated again to 230 °C or 240 °C all at 10 °C/min. Nitrogen was used as the purge gas, at a flow rate of 50 cm³/min.

### Results

Results from the TGA/DSC and DSC assessments are shown in Table 6.

TABLE 6
TGA/DSC data for AP1189 salts and AP1189 freebase.

| Counter ion | Solvent | TGA/DSC results |
|---|---|---|
| p- | 2-propanol: | Endothermic onset |
| Toluenesulfonic | water 90:10 | 234 °C |
| acid | v/v/ | |
| | methanol, | |
| | Pattern 1 | |
| Fumaric acid | 2-propanol: | Endothermic onset |
| | water 90:10 | 215 °C |
| | v/v, | |
| | Pattern 1 | |
| AP1189 | DCM | Endothermic onset |
| freebase | | 181 °C |
| Pattern 1 | | |
| AP1189 | DMSO | Endothermic onset |
| freebase | solvate | 103 °C |
| Pattern 3 | | |
| AP1189 | NMP | Endothermic onset |
| freebase | solvate | 83 °C |
| Pattern 5 | | |

### Example 5: Nuclear Magnetic Resonance

### Methods

NMR experiments were performed on a Bruker AVIIIHD spectrometer equipped with a DCH or PRODIGY cryoprobe operating at 500.12 or 500.23 MHz for protons. Experiments were performed in deuterated DMSO and each sample was prepared to ca. 10 mM concentration.

### Results

Chemical shifts and integration of ¹H-NMR signals from AP1189 salts are given in Table 7.

TABLE 7
Chemical shifts and integration of ¹H-NMR signals from
AP1189 salts and AP1189 freebase. In DMSO-d₆.

| | ¹H-NMR data | |
|---|---|---|
| AP1189 | Chemical shift | |
| salt | (ppm) | Integration |
| p-Toluenesulfonic acid | | |
| | 11.07 | 0.97 |
| | 8.16 | 1.00 |
| | 7.92 | 1.08 |
| | 7.82 | 1.08 |
| | 7.74 | 1.09 |
| | 7.66 | 1.11 |
| | 7.49 | 2.32 |
| | 7.37 | 3.26 |
| | 7.15 | 3.29 |
| | 6.80 | 1.05 |
| | 6.59 | 1.11 |
| | 6.34 | 2.04 |
| | 2.28 | 3.18 |
| | 2.04 | 0.16 |
| | 1.87 | 0.05 |
| | 1.27 | 0.77 |
| | 0.85 | 0.11 |
| Fumaric acid | | |
| | 13.34 | 1.42 |
| | 8.17 | 1.00 |
| | 7.90 | 1.37 |
| | 7.80 | 1.18 |
| | 7.73 | 1.14 |
| | 7.65 | 1.23 |
| | 7.08 | 0.92 |
| | 6.76 | 0.90 |
| | 6.52 | 0.91 |
| | 6.46 | 1.73 |
| | 6.41 | 0.94 |
| | 6.33 | 0.92 |
| | 2.09 | 0.17 |
| | 1.90 | 0.01 |
| | 1.24 | 0.16 |
| AP1189 freebase Pattern 1 | | |
| | 8.135 | 1.04 |
| | 7.879 | 1.10 |
| | 7.759 | 1.11 |
| | 7.623 | 1.09 |
| | 7.566 | 0.94 |
| | 6.937 | 0.89 |
| | 6.602 | 0.91 |
| | 6.485 | 0.95 |
| | 6.279 | 0.93 |
| | 6.109 | 1.00 |
| | 5.609 | 4.01 |
| | 4.047 | 0.19 |
| | 2.089 | 0.03 |
| | 1.987 | 0.27 |
| | 1.730 | 0.12 |
| | 1.177 | 0.28 |
| AP1189 DMSO solvate | | |
| | 8.141 | 1.13 |
| | 7.891 | 1.20 |
| | 7.763 | 1.19 |
| | 7.631 | 1.16 |
| | 7.565 | 1.02 |
| | 6.943 | 1.00 |
| | 6.594 | 1.01 |
| | 6.476 | 1.07 |
| | 6.282 | 1.00 |
| | 6.113 | 1.09 |
| | 5.703- | 4.20 |
| | 5.536 | |
| | 2.539 | 6.43 |
| AP1189 NMP solvate | | |
| | 8.137 | 1.14 |
| | 7.879 | 1.21 |
| | 7.759 | 1.20 |
| | 7.622 | 1.15 |
| | 7.563 | 1.03 |
| | 7.301 | 0.15 |
| | 6.979 | 0.15 |
| | 6.939 | 0.99 |
| | 6.852 | 0.13 |
| | 6.602 | 1.00 |
| | 6.481 | 1.05 |
| | 6.279 | 1.17 |
| | 6.107 | 1.07 |
| | 5.625 | 4.24 |
| | 3.297 | 2.56 |
| | 2.702 | 3.35 |
| | 2.180 | 2.30 |
| | 1.900 | 2.29 |

### Conclusion

Chemical shift values and integration of peaks corresponds to the expected salts or the free base of AP1189.

### Example 6: Solubility of AP1189 and Salts

### Methods

The solubility of AP1189 freebase, tosylate salt and fumarate salt were assessed in buffer solutions. An amount of the compound was suspended in buffer and agitated at ambient temperature for 24 hr, before the supernatant was sampled and analysed for the content of AP1189 by HPLC.

### Results

The results of the study are shown in table 8.

TABLE 8
Thermodynamic solubility results

| | | | | Salt or | |
|---|---|---|---|---|---|
| | | | | free- | Solu- |
| | | | | base | bility |
| | | | | input | at 24 h |
| Test | | | | concen- | (mM |
| com- | | | 24 h | tration | free- |
| pound | Buffer | Initial pH | pH | (mM) | base) |
| Freebase | 0.2 M HCl/ | 2.05 → 1.24 | 1.29 | 83* | >44.09 |
| Pattern 1 | KCl | 1.60 | 1.54 | | 13.87 |
| | FaSSGF | | | | |
| Tosylate | 0.2 M HCl/ | 1.22 | 1.26 | 31 | 0.31 |
| Salt | KCl | | | | |
| Pattern 1 | | | | | |
| Fumarate | 0.2 M HCl/ | 1.35 → 1.18 | 1.29 | 38 | 10.90 |
| Salt | KCl | | | | |
| Pattern 1 | | | | | |

→: pH adjusted using hydrochloric acid or sodium hydroxide
*: Estimated input concentration
FaSSGF: Fasted State Simulated Gastric Fluid

### Conclusion

The test compounds exhibited remarkably different solubilities at low pH. Specifically, the free base showed high solubility at pH 1.2, indicating the potential for using this compound in applications where a high solubility at low pH is desirable.

1. A crystalline form of 2-{3-[1-(2-nitrophenyl)-1H-pyrrol-2-yl]prop-2-en-1-ylidine}hydrazine-1-carboximidamide exhibiting at least X-ray lines (2-theta values) in a powder diffraction pattern when measured using Cu K_{α} radiation at 14.5±0.2, 23.9±0.2, and 26.7±0.2.

2. The crystalline form of 2-{3-[1-(2-nitrophenyl)-1H-pyrrol-2-yl]prop-2-en-1-ylidine}hydrazine-1-carboximidamide according to claim 1, further exhibiting one or more X-ray lines (2-theta values) in a powder diffraction pattern when measured using Cu K_{α} radiation selected from the group consisting of 11.6±0.2, 12.3±0.2, 13.4±0.2, 16.9±0.2, 17.6±0.2, 19.1±0.2, 19.7±0.2, 21.2±0.2, and 27.0±0.2.

3. The crystalline form of 2-{3-[1-(2-nitrophenyl)-1H-pyrrol-2-yl]prop-2-en-1-ylidine}hydrazine-1-carboximidamide according to any one of the preceding claims exhibiting an X-ray pattern (2-theta values) in a powder diffraction when measured using Cu K_{α} radiation according to FIG. 5.

4. A method for producing the 2-{3-[1-(2-nitrophenyl)-1H-pyrrol-2-yl]prop-2-en-1-ylidine}hydrazine-1-carboximidamide of the crystalline form according to any one of claims 1 to 3, said method comprising:
i. mixing 2-{3-[1-(2-nitrophenyl)-1H-pyrrol-2-yl]prop-2-en-1-ylidine}hydrazine-1-carboximidamide and a solvent to form a mixture; and
ii. isolating the 2-{3-[1-(2-nitrophenyl)-1H-pyrrol-2-yl]prop-2-en-1-ylidine}hydrazine-1-carboximidamide of the crystalline form according to any one of claims 1 to 3 from said mixture.

5. A method for producing the 2-{3-[1-(2-nitrophenyl)-1H-pyrrol-2-yl]prop-2-en-1-ylidine}hydrazine-1-carboximidamide of crystalline form according to any one of claims 1 to 3, said method comprising:
i. mixing a 2-{3-[1-(2-nitrophenyl)-1H-pyrrol-2-yl]prop-2-en-1-ylidine}hydrazine-1-carboximidamidium salt and a solvent to form a composition; and
ii. isolating the 2-{3-[1-(2-nitrophenyl)-1H-pyrrol-2-yl]prop-2-en-1-ylidine}hydrazine-1-carboximidamide of the crystalline form according to any one of claims 1 to 3 from said composition.

6. A method for producing the 2-{3-[1-(2-nitrophenyl)-1H-pyrrol-2-yl]prop-2-en-1-ylidine}hydrazine-1-carboximidamide of crystalline form according to any one of claims 1 to 3, said method comprising:
i. mixing 3-[1-(2-nitrophenyl)-1-H-pyrrole-2-yl]-propanal and amino guanidine or a salt thereof in a solvent to form a composition, and
ii. isolating the 2-{3-[1-(2-nitrophenyl)-1H-pyrrol-2-yl]prop-2-en-1-ylidine}hydrazine-1-carboximidamide of the crystalline form according to any one of claims 1 to 3 from said composition.

7. A method for producing the 2-{3-[1-(2-nitrophenyl)-1H-pyrrol-2-yl]prop-2-en-1-ylidine}hydrazine-1-carboximidamide of the crystalline form according to any one of claims 1 to 3, said method comprising:
i. providing a 2-{3-[1-(2-nitrophenyl)-1H-pyrrol-2-yl]prop-2-en-1-ylidine}hydrazine-1-carboximidamidium salt comprising 2-{3-[1-(2-nitrophenyl)-1H-pyrrol-2-yl]prop-2-en-1-ylidine}hydrazine-1-carboximidamide and a counter ion,
ii. separating the counter ion and 2-{3-[1-(2-nitrophenyl)-1H-pyrrol-2-yl]prop-2-en-1-ylidine}hydrazine-1-carboximidamide using ion exchange, and
iii. isolating 2-{3-[1-(2-nitrophenyl)-1H-pyrrol-2-yl]prop-2-en-1-ylidine}hydrazine-1-carboximidamide of the crystalline form according to any one of claims 1 to 3.

8. The method according to any one of claims 4 to 7, wherein the solvent is a protic or a polar aprotic solvent.

9. The method according to any one of claims 4 to 8, wherein the solvent is selected from the group consisting of 2-methyl tetrahydrofuran, 2-propanol, ethanol, water, acetone, acetonitrile, dichloromethane, dimethylsulfoxide, ethyl acetate, heptane, methanol, methylethyl ketone, methylisobutyl ketone, tert-butylmethyl ether, tetrahydrofuran, dimethylformamide (DMF), N-methyl-2-pyrrolidone (NMP), and toluene.

10. The method according to any one of claims 4 to 9, wherein the mixture or the composition is heated at least once before the isolating step.

11. The method according to any one of claims 4 to 10, wherein the mixture or the composition is heated and cooled in cycles for 15 min to 72 hours before the isolating step.

12. The method according to claim 11, wherein the heating is to about 40 °C, to about 60 °C, or to about 80 °C.

13. The method according to claim 11, wherein the cooling is to about 20 °C.

14. The method according to any one of claims 4 to 13, further comprising a step of adding an anti-solvent to the mixture or the composition before the isolation step.

15. The method according to claim 14, wherein the anti-solvent is a non-polar aprotic solvent.

16. The method according to claim 14, wherein the anti-solvent is water.

17. The method according to any one of claim 14, wherein the anti-solvent is selected from the group consisting of tert-butyl methyl ether, THF, and acetone.

18. The method according to any one of claims 4 to 17, wherein the isolation is carried out using filtration, centrifugation, and/or evaporation of the solvent or solvents.

19. The method according to claim 18, wherein the evaporation is carried out using spray drying, fluid bed drying, freeze drying, vacuum drying, tumble drying, rotary evaporation, and/or thin-film drying.

20. The method according to any one of claims 4 to 19, further comprising the step of adding a seed crystal of the crystalline form of 2-{3-[1-(2-nitrophenyl)-1H-pyrrol-2-yl]prop-2-en-1-ylidine}hydrazine-1-carboximidamide.

21. A crystalline form of 2-{3-[1-(2-nitrophenyl)-1H-pyrrol-2-yl]prop-2-en-1-ylidine}hydrazine-1-carboximidamide produced by the method of any one of claims 4 to 20.

22. A crystalline form of 2-{3-[1-(2-nitrophenyl)-1H-pyrrol-2-yl]prop-2-en-1-ylidine}hydrazine-1-carboximidamide which is a DMSO solvate exhibiting at least X-ray lines (2-theta values) in a powder diffraction pattern when measured using Cu K_{α} radiation at 16.7±0.2, 19.6±0.2, and 21.0±0.2.

23. The crystalline form of 2-{3-[1-(2-nitrophenyl)-1H-pyrrol-2-yl]prop-2-en-1-ylidine}hydrazine-1-carboximidamide DMSO solvate according to claim 22, further exhibiting one or more X-ray lines (2-theta values) in a powder diffraction pattern when measured using Cu K_{α} radiation selected from the group consisting of 14.3±0.2, 15.5±0.2, 18.4±0.2, and 23.5±0.2.

24. A crystalline form of 2-{3-[1-(2-nitrophenyl)-1H-pyrrol-2-yl]prop-2-en-1-ylidine}hydrazine-1-carboximidamide which is a DMF solvate exhibiting at least X-ray lines (2-theta values) in a powder diffraction pattern when measured using Cu K_{α} radiation at 23.2±0.2, 23.4±0.2, and 25.1±0.2.

25. The crystalline form of 2-{3-[1-(2-nitrophenyl)-1H-pyrrol-2-yl]prop-2-en-1-ylidine}hydrazine-1-carboximidamide DMF solvate according to claim 24, further exhibiting one or more X-ray lines (2-theta values) in a powder diffraction pattern when measured using Cu K_{α} radiation selected from the group consisting of 16.2±0.2, 21.0±0.2, and 22.9±0.2.

26. A crystalline form of 2-{3-[1-(2-nitrophenyl)-1H-pyrrol-2-yl]prop-2-en-1-ylidine}hydrazine-1-carboximidamide which is an NMP solvate exhibiting at least X-ray lines (2-theta values) in a powder diffraction pattern when measured using Cu K_{α} radiation at 16.2±0.2, 22.8±0.2, and 23.2±0.2.

27. The crystalline form of 2-{3-[1-(2-nitrophenyl)-1H-pyrrol-2-yl]prop-2-en-1-ylidine}hydrazine-1-carboximidamide NMP solvate according to claim 26, further exhibiting one or more X-ray lines (2-theta values) in a powder diffraction pattern when measured using Cu K_{α} radiation selected from the group consisting of 12.3±0.2, 15.5±0.2, 19.3±0.2, 20.1±0.2, and 20.8±0.2.

28. The crystalline form of 2-{3-[1-(2-nitrophenyl)-1H-pyrrol-2-yl]prop-2-en-1-ylidine}hydrazine-1-carboximidamide to any one of claims 1 to 3 exhibiting in differential scanning calorimetry an onset temperature between 174 and 188 °C using a heating rate of 10 °C per minute.

29. The crystalline form of 2-{3-[1-(2-nitrophenyl)-1H-pyrrol-2-yl]prop-2-en-1-ylidine}hydrazine-1-carboximidamide according to claim 28 exhibiting in differential scanning calorimetry an onset temperature of substantially 181 °C using a heating rate of 10 °C per minute.

30. The crystalline form of 2-{3-[1-(2-nitrophenyl)-1H-pyrrol-2-yl]prop-2-en-1-ylidine}hydrazine-1-carboximidamide DMSO solvate to any one of claims 22 to 23 exhibiting in differential scanning calorimetry an onset temperature between 96 and 110 °C using a heating rate of 10 °C per minute.

31. The crystalline form of 2-{3-[1-(2-nitrophenyl)-1H-pyrrol-2-yl]prop-2-en-1-ylidine}hydrazine-1-carboximidamide DMSO solvate according to claim 30 exhibiting in differential scanning calorimetry an onset temperature of substantially 103 °C using a heating rate of 10 °C per minute.

32. The crystalline form of 2-{3-[1-(2-nitrophenyl)-1H-pyrrol-2-yl]prop-2-en-1-ylidine}hydrazine-1-carboximidamide NMP solvate to any one of claims 26 to 27 exhibiting in differential scanning calorimetry an onset temperature between 76 and 90 °C using a heating rate of 10 °C per minute.

33. The crystalline form of 2-{3-[1-(2-nitrophenyl)-1H-pyrrol-2-yl]prop-2-en-1-ylidine}hydrazine-1-carboximidamide NMP solvate according to claim 32 exhibiting in differential scanning calorimetry an onset temperature of substantially 83 °C using a heating rate of 10 °C per minute.

34. A pharmaceutical composition comprising the crystalline form of 2-{3-[1-(2-nitrophenyl)-1H-pyrrol-2-yl]prop-2-en-1-ylidine}hydrazine-1-carboximidamide according to any one of claims 1 to 3 21, and 28 to 29.

35. A pharmaceutical composition comprising the crystalline form of 2-{3-[1-(2-nitrophenyl)-1H-pyrrol-2-yl]prop-2-en-1-ylidine}hydrazine-1-carboximidamide DMSO solvate according to any one of claims 22 to 23 and 30 to 31, the crystalline form of 2-{3-[1-(2-nitrophenyl)-1H-pyrrol-2-yl]prop-2-en-1-ylidine}hydrazine-1-carboximidamide DMF solvate according to any one of claims 24 to 25, or the crystalline form of 2-{3-[1-(2-nitrophenyl)-1H-pyrrol-2-yl]prop-2-en-1-ylidine}hydrazine-1-carboximidamide NMP solvate according to any one of claims 26 to 27 and 32 to 33, optionally further comprising a pharmaceutically acceptable excipient.

36. A pharmaceutical composition comprising the crystalline form of 2-{3-[1-(2-nitrophenyl)-1H-pyrrol-2-yl]prop-2-en-1-ylidine}hydrazine-1-carboximidamide according to any one of claims 1 to 3, 21, and 28 to 29 and a pharmaceutically acceptable excipient.

37. A pharmaceutical composition prepared by mixing the crystalline form of 2-{3-[1-(2-nitrophenyl)-1H-pyrrol-2-yl]prop-2-en-1-ylidine}hydrazine-1-carboximidamide according to any one of claims 1 to 3, 21, and 28 to 29 in a pharmaceutically acceptable solvent.

38. The pharmaceutical composition according to any one of claims 34 to 37, wherein the pharmaceutical composition is for oral administration.

39. A method of preparing a pharmaceutical composition, said method comprising mixing the crystalline form of 2-{3-[1-(2-nitrophenyl)-1H-pyrrol-2-yl]prop-2-en-1-ylidine}hydrazine-1-carboximidamide according to any one of 1 to 3, 21, and 28 to 29 and a pharmaceutically acceptable excipient.

40. A liquid formulation prepared from mixing the crystalline form of 2-{3-[1-(2-nitrophenyl)-1H-pyrrol-2-yl]prop-2-en-1-ylidine}hydrazine-1-carboximidamide according to any one of claims 1 to 3, 21, and 28 to 29 and a solvent.

41. A liquid formulation comprising the crystalline form of 2-{3-[1-(2-nitrophenyl)-1H-pyrrol-2-yl]prop-2-en-1-ylidine}hydrazine-1-carboximidamide according to any one of claims 1 to 3, 21, 28 to 29 and a solvent.

42. A method of preparing a liquid formulation, said method comprising mixing the crystalline form of 2-{3-[1-(2-nitrophenyl)-1H-pyrrol-2-yl]prop-2-en-1-ylidine}hydrazine-1-carboximidamide according to any one of claims 1 to 3, 21, and 28 to 29 and a solvent.

43. A method of preparing an amorphous form of 2-{3-[1-(2-nitrophenyl)-1H-pyrrol-2-yl]prop-2-en-1-ylidine}hydrazine-1-carboximidamide, said method comprising converting the crystalline form of 2-{3-[1-(2-nitrophenyl)-1H-pyrrol-2-yl]prop-2-en-1-ylidine}hydrazine-1-carboximidamide according to any one of claims 1 to 3, 21, and 28 to 29 to the amorphous form of 2-{3-[1-(2-nitrophenyl)-1H-pyrrol-2-yl]prop-2-en-1-ylidine}hydrazine-1-carboximidamide.

44. An amorphous form of 2-{3-[1-(2-nitrophenyl)-1H-pyrrol-2-yl]prop-2-en-1-ylidine}hydrazine-1-carboximidamide obtained by the method of claim 43.

45. A pharmaceutical composition comprising 2-{3-[1-(2-nitrophenyl)-1H-pyrrol-2-yl]prop-2-en-1-ylidine}hydrazine-1-carboximidamide in a form according to any one of claims 1 to 3, 21 to 33, and 44.

46. A unit dosage form of the pharmaceutical composition according to any of the preceding claims.

47. An oral formulation comprising 2-{3-[1-(2-nitrophenyl)-1H-pyrrol-2-yl]prop-2-en-1-ylidine}hydrazine-1-carboximidamide in a form according to any one of claims 1 to 3, 21 to 33, and 44.

48. The oral formulation according to claim 47 further comprising at least one pharmaceutically acceptable excipient.

49. The oral formulation according to any one of claims 47 and 48, wherein said 2-{3-[1-(2-nitrophenyl)-1H-pyrrol-2-yl]prop-2-en-1-ylidine}hydrazine-1-carboximidamide has a solubility of at least 15 mM at a pH of about 1.2, such as at least 20 mM at a pH of about 1.2, such as at least 25 mM at a pH of about 1.2, such as at least 30 mM at a pH of about 1.2, such as at least 35 mM at a pH of about 1.2, such as at least 40 mM at a pH of about 1.2.

50. The oral formulation according to any one of claims 47 to 49, wherein said 2-{3-[1-(2-nitrophenyl)-1H-pyrrol-2-yl]prop-2-en-1-ylidine}hydrazine-1-carboximidamide has a solubility of at least 10 mM at a pH of about 4.5, such as at least 15 mM at a pH of about 4.5, such as at least 20 mM at a pH of about 4.5.

51. The oral formulation according to any one of claims 47 to 50, wherein said oral formulation delivers and/or releases said 2-{3-[1-(2-nitrophenyl)-1H-pyrrol-2-yl]prop-2-en-1-ylidine}hydrazine-1-carboximidamide to the gastric compartment (or stomach), such as primarily or predominantly delivers and/or releases said 2-{3-[1-(2-nitrophenyl)-1H-pyrrol-2-yl]prop-2-en-1-ylidine}hydrazine-1-carboximidamide to the gastric compartment (or stomach).

52. The oral formulation according to any one of claims 47 to 51, wherein not less than about 65% to about 80% of said 2-{3-[1-(2-nitrophenyl)-1H-pyrrol-2-yl]prop-2-en-1-ylidine}hydrazine-1-carboximidamide is released in the gastric compartment.

53. The oral formulation according to any one of claims 47 to 52, wherein said 2-{3-[1-(2-nitrophenyl)-1H-pyrrol-2-yl]prop-2-en-1-ylidine}hydrazine-1-carboximidamide is released in the gastric compartment for gastric absorption.

54. The oral formulation according to any one of claims 47 to 53, wherein said oral formulation is a solid oral formulation.

55. The oral formulation according to any one of claims 47 to 54, wherein said oral formulation is a solid oral dosage form.

56. The oral formulation according to any one of claims 47 to 55, wherein said oral formulation is a tablet.

57. The oral formulation according to any one of claims 47 to 56, wherein not less than about 65% to about 80% of said 2-{3-[1-(2-nitrophenyl)-1H-pyrrol-2-yl]prop-2-en-1-ylidine}hydrazine-1-carboximidamide is dissolved into solution in the gastric compartment; such as wherein not less than about 65%, such as not less than about 70%, such as not less than about 75%, such as not less than about 80%, such as not less than about 85%, such as not less than about 90%, such as not less than about 95% of said 2-{3-[1-(2-nitrophenyl)-1H-pyrrol-2-yl]prop-2-en-1-ylidine}hydrazine-1-carboximidamide is dissolved into solution in the gastric compartment.

58. The oral formulation according to any one of claims 47 to 57, wherein said oral formulation is a delayed release formulation comprising said 2-{3-[1-(2-nitrophenyl)-1H-pyrrol-2-yl]prop-2-en-1-ylidine}hydrazine-1-carboximidamide targeting release in the gastric compartment.

59. The oral formulation according to any one of claims 47 to 58, wherein said oral formulation is a gastric-retention delayed release formulation comprising said 2-{3-[1-(2-nitrophenyl)-1H-pyrrol-2-yl]prop-2-en-1-ylidine}hydrazine-1-carboximidamide.

60. The oral formulation according to any one of claims 47 to 59, wherein said oral formulation is an immediate release formulation comprising said 2-{3-[1-(2-nitrophenyl)-1H-pyrrol-2-yl]prop-2-en-1-ylidine}hydrazine-1-carboximidamide targeting immediate release in the gastric compartment.

61. The oral formulation according to any one of claims 47 to 60, wherein not less than about 65% to about 80% of said 2-{3-[1-(2-nitrophenyl)-1H-pyrrol-2-yl]prop-2-en-1-ylidine}hydrazine-1-carboximidamide is dissolved into solution in about 10 minutes at a pH of about 1 to about 3; such as at a pH of about 1.2.

62. The oral formulation according to any one of claims 47 to 61, wherein not less than about 80% of said 2-{3-[1-(2-nitrophenyl)-1H-pyrrol-2-yl]prop-2-en-1-ylidine}hydrazine-1-carboximidamide is dissolved into solution in about 5 minutes at a pH of about 1 to about 3; such as at a pH of about 1.2.

63. The oral formulation according to any one of claims 47 to 62, wherein the disintegration time of said solid oral formulation is from % minute to 10 minutes, such as % minute to 1 minute, such as 1 to 2 minutes, such as 2 to 3 minutes, such as 3 to 4 minutes, such as 4 to 5 minutes, such as 5 to 6 minutes, such as 6 to 7 minutes, such as 7 to 8 minutes, such as 8 to 9 minutes, such as 9 to 10 minutes.

64. The oral formulation according to any one of claims 47 to 63, wherein said oral formulation comprises said 2-{3-[1-(2-nitrophenyl)-1H-pyrrol-2-yl]prop-2-en-1-ylidine}hydrazine-1-carboximidamide at a dosage from about 25 mg to about 650 mg, such as about 25 mg AP1189 free base, such as about 50 mg, such as about 100 mg, such as about 150 mg, such as about 200 mg, such as about 250 mg, such as about 300 mg, such as about 350 mg, such as about 400 mg, such as about 450 mg, such as about 500 mg, such as about 550 mg, such as about 600 mg, such as about 650 mg.

65. The crystalline form of 2-{3-[1-(2-nitrophenyl)-1H-pyrrol-2-yl]prop-2-en-1-ylidine}hydrazine-1-carboximidamide in a form according to any one of claims 1 to 3, 21 to 33, and 44, or the pharmaceutical composition according to any one of claims 34 to 43, and 45 to 64 for use in medicine.

66. The crystalline form of 2-{3-[1-(2-nitrophenyl)-1H-pyrrol-2-yl]prop-2-en-1-ylidine}hydrazine-1-carboximidamide according to any one of claims 1 to 3, 21 to 33, and 44, or the pharmaceutical composition according to any one of claims 34 to 43, and 45 to 64 for use in the treatment of a kidney disease, an arthritic disease, a cardiovascular disease, atherosclerosis, a viral disease or disorder, or a systemic inflammatory disorder.

67. The use according to claim 66, wherein the kidney disease is selected from the group consisting of:
i. kidney disease presenting with proteinuria,
ii. proteinuric kidney disease,
iii. glomerular disease,
iv. nephrotic syndrome (glomerulonephrosis),
v. primary nephrotic syndrome (primary glomerulonephrosis),
vi. secondary nephrotic syndrome (secondary glomerulonephrosis),
vii. an inflammatory kidney disease,
viii. glomerulonephritis (GN), and
ix. idiopathic membranous nephropathy (iMN).

68. The use according to claim 67, wherein said primary nephrotic syndrome is membranous glomerulonephritis (MGN) (or membranous nephropathy (MN)), focal segmental glomerulosclerosis (FSGS), membranoproliferative glomerulonephritis (MPGN) (mesangiocapillary glomerulonephritis), rapidly progressive glomerulonephritis (RPGN) (crescentic GN), or minimal change disease (MCD).

69. The use according to claim 67, wherein said secondary nephrotic syndrome is caused by an underlying autoimmune disease, an underlying cancer disease, an underlying genetic disorder, or by an underlying disease selected from the group consisting of: Systemic lupus erythematosus (SLE), Diabetic nephropathy, Sarcoidosis, Sjögren's syndrome, Amyloidosis, Multiple myeloma, Vasculitis, Cancer and Genetic disorders (such as congenital nephrotic syndrome).

70. The use according to claim 67, where said secondary nephrotic syndrome is caused by an infection, such as a urinary tract infection, such as an infection selected from the group consisting of HIV, syphilis, hepatitis such as hepatitis A, B and C, post-streptococcal infection, urinary schistosomiasis and Ebola. In some embodiments said secondary nephrotic syndrome is drug-induced.

71. The use according to claim 67, wherein said glomerulonephritis is selected from the group consisting of IgA nephropathy (Berger's disease), IgM nephropathy, Post-infectious glomerulonephritis and thin basement membrane disease.

72. The use according to claim 66, wherein the arthritic disease is selected from the group consisting of:
i. an auto-immune disease and/or an inflammatory disease that presents with joint inflammation,
ii. inflammatory arthritis,
iii. degenerative arthritis,
iv. metabolic arthritis,
v. reactive arthritis,
vi. infectious arthritis, and
vii. arthritis as part of a systemic inflammatory disease.

73. The use according to claim 72, wherein the inflammatory arthritis is selected from the group consisting of Rheumatoid Arthritis (RA), Psoriatic Arthritis, and Ankylosing Spondylitis.

74. The use according to claim 73, wherein the rheumatoid arthritis is juvenile rheumatoid arthritis (JRA).

75. The use according to claim 72, wherein the degenerative arthritis is osteoarthritis.

76. The use according to claim 72, wherein the metabolic arthritis is gouty arthritis.

77. The use according to claim 72, wherein the reactive and/or infectious arthritis is arthritis associated with infection with one or more of Hepatitis C, Chlamydia, gonorrhoea, salmonella or shigella.

78. The use according to claim 72, wherein the inflammatory disease is selected from the group consisting of systemic lupus erythematosus, mixed connective tissue disease, Still's disease, and Polymyalgia Rheumatica.

79. The use according to claim 72, wherein the viral disease or disorder is selected from the group consisting of:
i. a symptomatic viral disease or disorder,
ii. a symptomatic viral disease or disorder with inflammation,
iii. an inflammatory viral disease or disorder,
iv. a viral respiratory infection,
v. a viral respiratory disease or disorder,
vi. a viral disease or disorder of the lung,
vii. a viral disease or disorder with inflammation in the respiratory system,
viii. a viral disease or disorder with one or more respiratory symptoms,
ix. severe disease,
x. critical disease,
xi. viral pneumonia,
xii. viral bronchiolitis,
xiii. viral diseases or disorders with respiratory failure,
xiv. acute respiratory distress syndrome (ARDS),
xv. viral acute respiratory distress syndrome (ARDS),
xvi. symptomatic COVID-19 with acute respiratory distress syndrome (ARDS),
xvii. a viral disease or disorder with systemic inflammatory distress syndrome (SIDS) and/or sepsis,
xviii. a viral disease or disorder with pulmonary insufficiency,
xix. a viral disease or disorder with cytokine release syndrome (CRS) and/or a cytokine storm (hypercytokinemia),
xx. a viral disease or disorder caused by a viral infection selected from the group consisting of Severe Acute Respiratory Syndrome CoronaVirus 2 (SARS-CoV-2); SARS-CoV, MERS-CoV, the dengue virus and influenza virus (including Type A, Type B and Type C).

80. The use according to claim 79, wherein the inflammation is hyperinflammation, such as inflammation or hyperinflammation in one or more organs; such as one or more organs is selected from the group consisting of lungs, the respiratory tract, kidney, liver, pancreas, spleen, exocrine glands, endocrine glands, lymph nodes, brain, heart, muscles, bone marrow, skin, skeleton, bladder, reproduction organs including the phallopian tubes, eye, ear, vascular system, the gastroinstestinal tract including small intestines, colon, rectum, canalis analis and the prostate gland.

81. The use according to claim 79, wherein the viral respiratory infection is viral lower respiratory infection.

82. The use according to claim 79, wherein the viral disease or disorder with inflammation in the respiratory system is a viral disease or disorder with inflammation in the respiratory system in the lungs and/or respiratory tract.

83. The use according to claim 79, wherein said one or more respiratory symptoms are selected from the group consisting of cough, dry cough, dyspnea, impaired oxygenation, respiratory illness, respiratory dysfunction, respiratory failure, respiratory syndrome and acute respiratory disease (ARD).

84. The use according to claim 79, wherein said severe disease present with dyspnoea, increased respiratory frequency, reduced blood oxygen saturation and/or lung infiltrates.

85. The use according to claim 79, wherein said critical disease present with respiratory failure, septic shock, and/or multiple organ dysfunction (MOD) or multiple organ failure (MOF).

86. The use according to claim 66, wherein the cardiovascular disease is selected from the group consisting of:
i. coronary artery diseases (CAD),
ii. stroke,
iii. heart failure,
iv. hypertensive heart disease,
v. rheumatic heart disease,
vi. cardiomyopathy,
vii. abnormal heart rhythms,
viii. congenital heart disease,
ix. valvular heart disease,
x. carditis,
xi. aortic aneurysms,
xii. peripheral artery disease,
xiii. vascular disease,
xiv. thromboembolic disease,
xv. venous thrombosis,
xvi. vascular inflammation, and
xvii. atherosclerotic cardiovascular disease.

87. The use according to claim 86, wherein the coronary artery diseases (CAD) is selected from the group consisting of angina and myocardial infarction.

88. The use according to claim 86, wherein the atherosclerotic cardiovascular disease is selected from the group consisting of coronary artery disease, stroke (cerebrovascular disease), and peripheral artery disease.

89. The use according to claim 86, wherein the systemic inflammatory disorder is an autoimmune disorder.

90. The use according to claim 86, wherein the systemic inflammatory disorder is selected from the group consisting of Behçet disease, sarcoidosis, systemic lupus erythematosus, juvenile idiopathic arthritis, scleroderma, Sjögren syndrome, myositis including dermamyositis and polymyositis, vasculitis, giant cell arteritis, ankylosing spondylitis, polymyalgia rheumatic and psoriatic arthritis.

91. A method of treating a disease or disorder in a subject in need thereof, said method comprising administering the crystalline form of 2-{3-[1-(2-nitrophenyl)-1H-pyrrol-2-yl]prop-2-en-1-ylidine}hydrazine-1-carboximidamide according to any one of claims 1 to 3, 21 to 33, and 44, or the pharmaceutical composition according to any one of claims 34 to 43, and 45 to 64 to a subject in need thereof.

92. Use of the crystalline form of 2-{3-[1-(2-nitrophenyl)-1H-pyrrol-2-yl]prop-2-en-1-ylidine}hydrazine-1-carboximidamide according to any one of claims 1 to 3, 21 to 33, and 44, or the pharmaceutical composition according to any one of claims 34 to 43, and 45 to 64 for the manufacture of a medicament for treatment of a disease or disorder.
